(19) United States
(12) Patent Application Publication
(10) Pub. No.: US 2003/0032617 A1
(43) Pub. Date: Feb. 13, 2003

(54) Composition and methods for the treatment of skin disorders

(76) Inventors: Avikam Harel, Tel Aviv (IL); Olga Bloch, Petach Tikva (IL)

Correspondence Address: G.E. EHRLICH (1995) LTD., c/o ANTHONY CASTORINA, 2001 JEFFERSON DAVIS HIGHWAY, SUITE 207, ARLINGTON, VA, 22202, US

(21) Appl. No.: 10/192,842

(22) Filed: Jul. 11, 2002

Related U.S. Application Data
(63) Continuation-in-part of application No. PCT/IL01/00017, filed on Jan. 9, 2001.

(30) Foreign Application Priority Data
Jan. 11, 2000 (IL) 133976

(51) Int. Cl. A61K 31/7076; A61K 31/59; A61K 31/44

(52) U.S. Cl. 514/46; 514/167; 514/168; 514/356

(57) ABSTRACT

Methods and pharmaceutical compositions for use in the treatment of a benign and/or a malignant proliferative pathologies are disclosed. The methods comprise administration of nicotinamide or its analogs and/or cADPR or its analogs, optionally in combination with a vitamin D3 analog or a Vitamin A analog.

This Application is a Continuation-In-Part of PCT/IL01/00017, filed Jan. 9, 2001, which claims the benefit of priority from IL 133976, filed Jan. 11, 2000.

### FIELD AND BACKGROUND OF THE INVENTION

The present invention relates to compositions, kits and methods for treating skin disorders and, more particularly, to compositions, kits and methods for treating hyperproliferative epidermal pathologies and other conditions of the epidermis.

Normal growth and differentiation of epidermal cells requires a number of regulating factors such as Vitamin D3, Vitamin A, a number of cytokines and growth factors and extra- and intercellular free Ca⁺². Skin benign and malignant hyperproliferative disorders arise from faulty regulation of growth and differentiation of epidermal cells. The faulty regulation is often caused either by lack of response or lack of appropriate response to regulating factors, or due to abnormal levels or function of the regulating factors themselves. For example, it is well known that inappropriate growth and differentiation of epidermal cells results from aberrant signaling through the epidermal growth factor receptor. This abnormality may contribute to the development of various skin disorders such as psoriasis, ichythyiosis, squamous cell carcinomas and multiple human tumors. Hence, controlling the differentiation and/or proliferation of epidermal cells is advantageous in treating hyperproliferative skin disorders.

Many compositions for treating skin disorders are known. For example, there are many reports of successful treatment of psoriasis and other related skin disorders in humans, following oral or topical treatment with vitamin D3 and its analogues [6].

It is well known that Ca⁺²-signaling pathways are involved in differentiation of keratinocytes. Recently, cyclic ADP ribose (cADPR), which is a cyclic derivative of NAD+, was discovered as a potent Ca⁺²-mobilizing natural compound in different eukaryotic cells [7]. However, the effect of cADPR on Ca¹²-signaling in human keratinocytes and hence its effect on proliferation and/or differentiation of human epidermal cells has not been studied yet.

Recently it has been shown that all-trans-retinoic acid (atRA), which is a vitamin A metabolite, induces activation of cap-ribose synthesis in renal LLC-PK₁ cells by enhancing activity of ADPR-cycles without affecting of ADPR-hydrolase [8]. However the effect of atRA on cADPR synthesis in human keratinocytes as well as its effect on proliferation and/or differentiation of human epidermal cells has not been studied yet.

Nicotinamide (NA) is a water-soluble derivative of vitamin B, whose physiological active forms are nicotinamide adenine dinucleotide (NAD+/NADII) and nicotinamide adenine dinucleotidc phosphate (NADP+/NADPH). The physiological active forms of NA serve as coenzyme in a variety of important metabolic reactions.

Recently, it has been shown that nicotinamide (NA), can induce differentiation of insulin-producing cells [1]. Successful treatment and prevention of insulin dependent diabetes mellitus with NA was demonstrated in animal models [2] and therapeutic and prophylactic effects of NA on diabetes mellitus are now in the phase of international clinical trials [3].

NA is also known as a weak free-radical scavenger, inhibitor of poly-ADP-ribose synthetase and inducible nitric oxide synthase in pancreatic islets [4]. There is a single report about the significant role of NA in cellular NAD regeneration after peroxide-induced depletion [5].

U.S. Pat. No. 4,505,896 discloses compositions and methods for the treatment of acne vulgaris. The compositions disclosed in this patent include nicotinic acid or nicotinamide and, optionally, another chemical agents such as sulfur, salicylic acid and Vitamin A acid, which are known to be effective in treating acne. Nevertheless, the compositions and methods disclosed in U.S. Pat. No. 4,505,896 are specifically directed toward the treatment of acne vulgaris, which is an inflammatory disease and not a hyperproliferative benign (e.g., psoriasis) or malignant skin disorders.

U.S. Pat. No. 6,248,763 discloses compositions for treating skin conditions, which include derivatives of nicotinic acid or nicotinamide and, in particular, methyl nicotinate, as the active ingredient. These compositions are topically applies and are directed toward the treatment of acne and other skin conditions such as fine lines and age spots, bums, etc. However, like U.S. Pat. No. 4,505,896, U.S. Pat. No. 6,248,763 fails to teach compositions and methods for the treatment of hyperproliferative skin disorders.

Hence, although the prior art teaches various roles and uses of NA and/or combinations thereof with various agents, the prior art clearly fails to teach the effects of NA on epidermal cells and, in particular, on the proliferation and differentiation of these cells and hence fails to teach uses of NA or its derivatives in the treatment of hyperproliferative skin disorders and as an anti-oxidant in epidermal cells.

The prior art further fails to teach uses of Vitamin D3, atRA and cADPR and their agonistic derivatives in the treatment of hyperproliferative skin disorders.

### SUMMARY OF THE INVENTION

While conceiving the present invention, it was hypothesized that nicotinamide and/or other agents that are known to be associated with cell differentiation and/or proliferation, such as cADPR, can exert anti-proliferative effects in various epidermal cell associated pathologies.

While reducing the present invention to practice, it was surprisingly found that (i) both nicotinamide and cADPR promote the differentiation and inhibit the proliferation of benign and malignant epidermal cells; (ii) combinations of nicotinamide and agents such as cADPR and metabolites of Vitamins A and D3 exert synergistic effect on epidermal cell proliferation; and (iii) nicotinamide is highly effective as an anti-oxidant against auto-oxidative agents.

Hence, according to one aspect of the present invention there is provided a method of treating a benign or malignant hyperproliferative epidermal pathology in a subject in need thereof The method comprises administering to the subject a therapeutically effective amount of an agent selected from the group consisting of nicotinamide, a nicotinamide agonist, a nicotinamide derivative, a nicotinamide metabolite and prodrugs thereof.

According to another aspect of the present invention there is provided another method treating a benign or malignant hyperproliferative epidermal pathology in a subject in need thereof. The method comprises administering to the subject a therapeutically effective amount of an agent selected from the group consisting of cyclic adenosine diphosphate-ribose (cADPR), a cADPR derivative, a cADPR metabolite, a cADPR agonist and prodrugs thereof.

As is described hereinabove, it was found that the administration of nicotinamide in combination with cADPR results in a synergistic effect with respect to inhibiting proliferation of epidermal cells, and hence, according to yet another aspect of the present invention there is provided a method of treating a benign or malignant hyperproliferative epidermal pathology in a subject in need thereof. This method comprises administering to the subject a therapeutically effective amount of an agent selected from the group consisting of nicotinamide, a nicotinamide agonist, a nicotinamide derivative, a nicotinamide metabolite and prodrugs thereof, in combination with a therapeutically effective amount of an agent selected from the group consisting of cyclic adenosine diphosphate-ribose (cADPR), a cADPR derivative, a cADPR metabolite, a cADPR agonist and prodrugs thereof.

Each of the above methods can further comprise administering to the subject, in combination with the agent(s) described above, a therapeutically effective amount of an agent selected from the group consisting of Vitamin D3, a Vitamin D3 metabolite, a Vitamin D3 agonist, a Vitamin D3 derivative and prodrugs thereof.

Alternatively or additionally, each of the above methods can further comprise administering to the subject, in combination with the agent(s) described above, a therapeutically effective amount of an agent selected from the group consisting of Vitamin A, a Vitamin A metabolite, a Vitamin A agonist, a Vitamin A derivative and prodrugs thereof.

Hence, according to further aspects of the present invention, there are provided additional methods of treating a benign or malignant hyperproliferative epidermal pathology in a subject in need thereof. These methods comprise administering to the subject a therapeutically effective amount of an agent selected from the group consisting of nicotinamide, a nicotinamide agonist, a nicotinamide derivative, a nicotinamide metabolite and prodrugs thereof, or a therapeutically effective amount of an agent selected from the group consisting of cyclic adenosine diphosphate-ribose (cADPR), a cADPR derivative, a cADPR metabolite, a cADPR agonist and prodrugs thereof, in combination with a therapeutically effective amount of an agent selected from the group consisting of Vitamin D3, a Vitamin D3 metabolite, a Vitamin D3 agonist, a Vitamin D3 derivative and prodrugs thereof, or in combination with a therapeutically effective amount of an agent selected from the group consisting of Vitamin A, a Vitamin A metabolite, a Vitamin A agonist, a Vitamin A derivative and prodrugs thereof.

According to further aspects of the present invention there are provided pharmaceutical, cosmetic or cosmeceutical compositions, identified for use in the treatment of a benign or malignant hyperproliferative epidermal pathology and/or for use in the treatment of a condition whereby increasing anti-oxidative properties of epidermal cells is advantageous. Such a condition can be, for example, aging or cancer.

The pharmaceutical, cosmetic or cosmeceutical compositions of the present invention comprise, as an active ingredient or as a combination of active ingredients, a therapeutically effective amount of an agent selected from the group consisting of nicotinamide, a nicotinamide agonist, a nicotinamide derivative, a nicotinamide metabolite and prodrugs thereof, and/or a therapeutically effective amount of an agent selected from the group consisting of cyclic adenosine diphosphate-ribose (cADPR), a cADPR derivative, a cADPR metabolite, a cADPR agonist and prodrugs thereof; and a pharmaceutically, cosmetically or cosmeceutically acceptable carrier.

Each of the above pharmaceutical, cosmetic or cosmeceutical compositions can further comprise, in combination with the agent(s) described above, a therapeutically effective amount of an agent selected from the group consisting of Vitamin D3, a Vitamin D3 metabolite, a Vitamin D3 agonist, a Vitamin D3 derivative and prodrugs thereof.

Alternatively or additionally, each of the above pharmaceutical compositions can further comprise, in combination with the agent(s) described above, a therapeutically effective amount of an agent selected from the group consisting of Vitamin A, a Vitamin A metabolite, a Vitamin A agonist, a Vitamin A derivative and prodrugs thereof.

In all the above pharmaceutical, cosmetic or cosmeceutical compositions, the therapeutically effective amount of nicotinamide preferably ranges between 1 mM and 50 mM, most preferably, between 1 mM and 10 mM.

The therapeutically effective amount of cADPR preferably ranges between 10 μM and 100 μM.

The pharmaceutical, cosmetic or cosmeceutical compositions of the present invention can optionally be packaged in a container and identified in print in or on the container, for use in the treatment of a benign and/or a malignant hyperproliferative epidermal pathology.

Alternatively, the pharmaceutical, cosmetic or cosmeceutical compositions of the present invention can be packaged in a container and identified in print in or on the container for use in the treatment of a condition whereby increasing anti-oxidative properties of epidermal cells is advantageous. Hence, according to further aspects of the present invention there are provided pharmaceutical, cosmetic or cosmeceutical kits, which comprise a therapeutically effective amount of an agent selected from the group consisting of nicotinamide, a nicotinamide agonist, a nicotinamide derivative, a nicotinamide metabolite and prodrugs thereof, and/or a therapeutically effective amount of an agent selected from the group consisting of cyclic adenosine diphosphate-ribose (cADPR), a cADPR derivative, a cADPR metabolite, a cADPR agonist and prodrugs thereof.

Each of the above pharmaceutical, cosmetic or cosmeceutical kits can further comprise a therapeutically effective amount of an agent selected from the group consisting of Vitamin D3, a Vitamin D3 metabolite, a Vitamin D3 agonist, a Vitamin D3 derivative and prodrugs thereof.

Alternatively or additionally, each of the above pharmaceutical, cosmetic or cosmeceutical kits can further comprise a therapeutically effective amount of an agent selected from the group consisting of Vitamin A, a Vitamin A metabolite, a Vitamin A agonist, a Vitamin A derivative and prodrugs thereof.

In each of the pharmaceutical, cosmetic or cosmeceutical kits of the present invention, the agents are packaged individually within the kit.

According to further aspects of the present invention, there are provided methods of increasing anti-oxidative properties of epidermal cells. These methods comprise contacting the cells with an effective amount of an agent selected from the group consisting of nicotinamide, a nicotinamide agonist, a nicotinamide derivative, a nicotinamide metabolite and prodrugs thereof and/or with an effective amount of an agent selected from the group consisting of cyclic adenosine diphosphate-ribose (cADPR), a cADPR derivative, a cADPR metabolite, a cADPR agonist and prodrugs thereof.

According to further features in preferred embodiments of the invention described below, the agent is nicotinamide and the effective amount ranges between 1 mM and 50 mM.

Other methods of increasing anti-oxidative properties of epidermal cells, according to the present invention, comprise contacting the cells with one of the agents described above, in combination with an effective amount of an agent selected from the group consisting of Vitamin D3, a Vitamin D3 metabolite, a Vitamin D3 agonist, a Vitamin D3 derivative and prodrugs thereof or an effective amount of an agent selected from the group consisting of Vitamin A, a Vitamin A metabolite, a Vitamin A agonist, a Vitamin A derivative and prodrugs thereof

According to still further aspects of the present invention, there are is provided methods of inhibiting proliferation of benign or malignant hyperproliferative epidermal cells. These method comprise contacting the cells with a therapeutically effective amount of an agent selected from the group consisting of nicotinamide, a nicotinamide agonist, a nicotinamide derivative, a nicotinamide metabolite and prodrugs thereof and/or with a therapeutically effective amount of an agent selected from the group consisting of cyclic adenosine diphosphate-ribose (cADPR), a cADPR derivative, a cADPR metabolite, a cADPR agonist and prodrugs thereof.

Other methods of inhibiting proliferation of benign or malignant hyperproliferative epidermal cells, according to the present invention, comprise contacting the cells with one of the agents described above, in combination with a therapeutically effective amount of a second agent selected from the group consisting of Vitamin D3, a Vitamin D3 metabolite, a Vitamin D3 agonist, a Vitamin D3 derivative and prodrugs thereof or a second agent selected from the group consisting of Vitamin A, a Vitamin A metabolite, a Vitamin A agonist, a Vitamin A derivative and prodrugs thereof

According to further features in preferred embodiments of the invention described below, the hyperproliferative benign epidermal pathology is selected from the group consisting of psoriasis, ichythyiosis, common warts, keratoacanthoma, seborrhoic keratosis and seborrhea.

According to still further features in the described preferred embodiments the hyperproliferative malignant epidermal pathology is selected from the group consisting of squamous-cell carcinoma (SCC), basal cell carcinoma (BCC) and a non-melanoma skin cancer (NMSC).

According to still further features in the described preferred embodiments the Vitamin D3 metabolite is 1α,25-dihydroxy-vitamin D3.

According to still further features in the described preferred embodiments, a therapeutically effective amount of 1α,25-dihydroxy-vitamin D3 ranges between 1 nM and 200 nM.

According to still further features in the described preferred embodiments the Vitamin A metabolite is an all-trans-retinoic acid.

According to still further features in the described preferred embodiments, a therapeutically effective amount of all-trans-retinoic acid ranges between 0.1 nM and 10 nM.

According to still further features in the described preferred embodiments the Vitamin A agonist is a retinoic acid receptor agonist.

The present invention successfully addresses the shortcomings of the presently known configurations by providing methods, pharmaceutical, cosmetic or cosmeceutical compositions and pharmaceutical, cosmetic or cosmeceutical kits for treating various benign and malignant proliferative pathologies and for increasing anti-oxidative properties of epidermal cells, using highly efficient agents or combinations of agents that exert synergistic effects.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, amid are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1(a-b) show the anti-proliferative effect of NA on HaCat and A431 cell proliferation (FIG. 1a) and on cultured human epidermal keratinocytes (FIG. 1b);

FIG. 2 shows the anti-proliferative effect of a D3 metabolite (1α25(OH)₂D3) on HaCat and A431 cell proliferation;

FIG. 3 shows the anti-proliferative effect of cADPR on HaCat and A431 cell proliferation;

FIG. 4 shows the anti-proliferative effect of a Vitamin A metabolite (atRA) on HaCat and A431 cell proliferation;

FIGS. 5(a-b) show the anti-proliferative effect of a combination of NA and a D3 metabolite (1α 25(OH)₂D3) on HaCat cell line proliferation (FIG. 5a) and the synergistic effect of this combination as compared with the anti-proliferative effects of each of these compounds separately (NA and 1α 25(OH)₂D3), on this cell line, shown as the effect of the combined treatment minus the effect of each of the compounds (FIG. 5b);

FIGS. 6(a-b) show the anti-proliferative effect of a combination of NA and a D3 metabolite (1α 25(OH)₂D3) on A431 cell line proliferation (FIG. 6a) and the synergistic effect of this combination as compared with the anti-proliferative effects of each of these compounds separately (NA and 1α 25(OH)₂D3), on this cell line, shown as the effect of the combined treatment minus the effect of each of the compounds (FIG. 6b);

FIGS. 7(a-b) show the anti-proliferative effect of a combination of NA and cADPR on IICat cell line proliferation (FIG. 7a) and the synergistic effect of this combination as compared with the anti-proliferative effects of each of these compounds separately (NA and cADPR), on this cell line, shown as the effect of the combined treatment minus the effect of each of the compounds (FIG. 7b);

FIGS. 8(a-b) show the anti-proliferative effect of a combination of NA and cADPR on A431 cell line proliferation (FIG. 7a) and the synergistic effect of this combination as compared with the anti-proliferative effects of each of these compounds separately (NA and cADPR), on this cell line, shown as the effect of the combined treatment minus the effect of each of the compounds (FIG. 7b);

FIGS. 9(a-b) show the anti-proliferative effect of a combination of NA and a Vitamin A metabolite (atRA) on HaCat cell line proliferation (FIG. 9a) and the synergistic effect of this combination as compared with the anti-proliferative effects of each of these compounds separately (NA and atRA), on this cell line, shown as the effect of the combined treatment minus the effect of each of the compounds (FIG. 9b);

FIGS. 10(a-b) show the anti-proliferative effect of a combination of NA and a Vitamin A metabolite (atRA) on A431 cell line proliferation (FIG. 10a) and the synergistic effect of this combination as compared with the anti-proliferative effects of each of these compounds separately (NA and atRA), on this cell line, shown as the effect of the combined treatment minus the effect of each of the compounds (FIG. 10b);

FIG. 11 shows the effect of NA on involucrin and keratin k10 expression in HaCat cells;

FIG. 12 shows the effect of NA on basal and envelope cornified cell expression in HaCat cell line;

FIG. 13 shows the effect of NA on apoptosis level in HaCat and A431 cell lines; and

FIG. 14 shows the resistance of HaCat cells treated for long-term period with NA to oxidative stress induced by hydrogen peroxide (H₂O₂).

### DESCRIPTION OF THE PREFERRED EMBODIMENTS

The present invention is of pharmaceutical, cosmetic or cosmeceutical compositions, pharmaceutical cosmetic or cosmeceutical kits and methods, which can be used in the treatment of skin disorders. Specifically, the present invention can be used in the treatment of benign and malignant proliferative epidermal pathologies, and in the treatment of conditions that require increasing anti-oxidative properties of epidermal cells, such as, for example, aging.

The principles and operation of the pharmaceutical, cosmetic or cosmeceutical compositions, pharmaceutical, cosmetic or cosmeceutical kits and methods according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

While conceiving the present invention, it was hypothesized that nicotinamide and/or other agents that are known to be associated with cell differentiation and/or proliferation, such as cADPR, can exert anti-proliferative effects in various epidermal cell associated pathologies. It was further hypothesized that using such agents in combination with other agents that are known to affect certain skin disorders, such as Vitamins D3 and A and their analogs, could result in enhanced anti-proliferative activity of the agents.

While reducing the present invention to practice, as is demonstrated in the Examples section that follows, it was surprisingly found, inter alia, that both nicotinamide and cADPR promote the differentiation and inhibit the proliferation of benign and malignant epidermal cells.

As is described in detail in the Examples section, the antiproliferative activity of these compounds was tested in two model systems: (i) a spontaneously immortalized human kcratinocyte, which is referred to herein as “HaCat cell line” or “HaCat cells”, and serves as a model for highly proliferative epidermis, such as, but not limited to, psoriatic epidermis [9], and as a model for effects of external modulators of epidermal differentiation [10]; and (ii) an epidermal carcinoma cell line, which is referred to herein as “A431 cell line” or “A431 cells”, which bear the mutated alleles of p53, and serves as a model for testing anti-cancerogenic drugs [11] and hence as a model for malignant hyperproliferative pathologies.

The antiproliferative effects of nicotinamide and cADPR were further demonstrated on rapidly proliferating human keratinocytes, which are referred to herein as “Cultured Human Epidermal Keratinocytes”, and serves as model for detecting antiproliferative treatment of psoriasis [12].

These models can be used, according to the present invention, to test the antiproliferative efficacy of agents which are assumed analogs of the agents described herein, such as their derivatives, metabolites, agonists and prodrugs.

The surprising findings obtained in these models with respect to the s effects of both nicotinamide and cADPR on the proliferation and differentiation of epidermal cells, clearly indicate that these compounds can serve as highly potent and versatile agents in the treatment of various hyperproliferative epidermal pathologies.

Hence, according to the present invention, there are provided methods of treating benign or malignant hyperproliferative epidermal pathologies in a subject in need thereof. These methods are effected by administering to the subject a therapeutically effective amount of a nicotinamide agent or a therapeutically effective amount of a cADPR agent, as these terms are defined hereinbelow.

The nicotinamide agent is either nicotinamide itself, or any nicotinamide analog that is known to act similarly thereto, such as, but not limited to, a nicotinamide agonist, a nicotinamide derivative or a nicotinamide metabolite. The nicotinamide agent can further be a prodrug of each of the nicotinamide agents described.

Similarly, the cADPR agent is either cADPR itself or any cADPR analog that is known to act similarly thereto, such as, but not limited to, a cADPR agonist, a cADPR derivative or a cADPR metabolite. The cADPR agent can further be a prodrug of each of the cADPR agents described.

As used herein, the phrase “hyperproliferative epidermal pathology” includes any disease, condition or syndrome that is characterized by a higher than normal level of proliferation of epidermal cells, and, as a rule, also by abnormal differentiation.

The hyperproliferative epidermal pathology may be malignant or benign, as is discussed hereinabove and is demonstrated in detail in the Examples section that follows.

Representative examples of malignant hyperproliferative epidermal pathologies that are treatable by the methods of the present invention include, without limitation, squamous-cell carcinoma (SCC), basal-cell carcinoma (BCC) and other non-melanoma skin cancers (NMSCs).

Representative examples of benign hyperproliferative epidermal pathologies that are treatable by the methods of the present invention include, without limitation, psoriasis, common warts, keratoacanthoma, seborrhoic keratosis, seborrhea and ichthyosis.

Herein, the term “treating” includes abrogating, substantially inhibiting, slowing or reversing the progression of a pathology, substantially ameliorating clinical symptoms of a pathology or substantially preventing the appearance of clinical symptoms of a pathology. These effects may be manifested, for example, by a decrease in the rate of proliferation, an improved differentiation or a combination thereof and/or by complete elimination of the abnormal proliferation and differentiation of the epidermal cells in the treated subject.

The term “administering” as used herein describes a method for bringing a nicotinamide agent, a cADPR agent, or any other agent or combination of agents described herein, and epidermal cells affected by the pathology together in such a manner that the agent can affect the proliferation and/or differentiation of these cells.

Preferably, the administration according to the present invention is accomplished either by topical application or by subcutaneous administration of the agent or the combination of agents.

The phrase “therapeutically effective amount”, as used herein, describes an amount administered to an individual, which is sufficient to abrogate, substantially inhibit, slow or reverse the progression of an epidermal pathology, to substantially ameliorate clinical symptoms of an epidermal pathology or substantially prevent the appearance of clinical symptoms of an epidermal pathology.

More specifically, the phrase “therapeutically effective amount” defined above, describes an amount of an agent or a combination of agents administered to an individual, which improves, in a measurable manner, the differentiation of the epidermal cells, a feature which is determined, for example, by the indirect immunofluorescence analysis of keratin 10 and involucrin expression and/or by determination of the level of envelope cornified formation [13]. Alternatively, this phrase describes an administered amount of an agent or a combination of agents which can decrease, to a measurable amount, the proliferation of the cells, a feature which is determined, for example, by measurement of the activity of mitochondrial dehydrogenase enzymes of living cells (MIT assay) [14] and by counting of basal cells level [15].

While reducing the present invention to practice it was further surprisingly found that (i) a synergistic anti-proliferative effect was exerted by a combination of nicotinamide and cADPR that was applied on the tested cell lines described hereinabove; and (ii) such a synergistic anti-proliferative effect was also exerted by combinations of nicotinamide and metabolites of Vitamin D3 or A.

These findings indicate that a combination of agents that are capable to modulate the proliferation and/or differentiation of epidermal cells, such as the combination of an NA agent and a cADPR agent, as well as combinations of each of these agents with agents that are known as useful in the treatment of skin disorders, such as analogs of Vitamin D3 and Vitamin A, are highly efficient in treating hyperproliferative epidermal pathologies.

Hence, according to another aspect of the present invention there are provided additional methods of treating benign or malignant hyperproliferative epidermal pathologies in subjects in need thereof.

In one particular, the method of treating a benign or malignant hyperproliferative epidermal pathology in a subject in need thereof is effected by administering to the subject a therapeutically effective amount of a nicotinamide agent, as described hereinabove, in combination with a therapeutically effective amount of a cADPR agent, as described hereinabove.

In another particular, the method of treating a benign or malignant hyperproliferative epidermal pathology in a subject in need thereof is effected by administering to the subject a therapeutically effective amount of a nicotinamide agent, as described hereinabove, in combination with a therapeutically effective amount of a Vitamin D3 agent.

The Vitamin D3 agent, according to the present invention, is any analog of Vitamin D3, such as, but not limited to, a Vitamin D3 metabolite, a Vitamin D3 agonist and a Vitamin D3 derivative. The Vitamin D3 agent can flushed be a prodrug of each of the above agents.

Preferably, this method of the present invention is effected by administering to the subject a therapeutically effective amount of nicotinamide, in combination with 1α,25-dihydroxy-Vitamin D3, which is also referred to herein as “1α,25(OH)₂D3”, and is known as a metabolite of Vitamin D3.

As is described hereinabove, Vitamin D3 and its analogs are known as useful agents in the treatment of psoriasis and other related skin diseases [6]. However, the synergistic anti-proliferative effect exerted by a Vitamin D3 metabolite when used in combination with NA has never been observed hitherto.

As is described in detail in the Examples section that follows, the above combination was found more effective in promoting differentiation and inhibiting proliferation of different epidermal cell lines, than the sum of each of the individual effects of NA and the vitamin D3 metabolite when administered separately. In fact, at the tested concentrations (1 nM-10000 nM), 1α,25-dihydroxy-Vitamin D3 was found inactive toward proliferation and/or differentiation of epidermal cells.

Other Vitamin D3 metabolites, as well as agonists, derivatives and prodrugs of Vitamin D3 are expected to act similarly when used in the context of the present invention.

Representative examples of other suitable metabolites of vitamin D3 which can be used in the context of present invention include, without limitation, 25-hydroxycholecalciferol (25 OH D3) and 24R, 25-dihydroxycholecalciferol (24R, 25(OH)₂D3).

Vitamin D3 agents are also expected to exert synergistic effect on the proliferation and/or differentiation of epidermal cells, when used in combination with cADPR and its related compounds described hereinabove.

In yet another particular, the method of this aspect of the present invention is effected by administering to the subject a therapeutically effective amount of a nicotinamide agent, as described hereinabove, in combination with a therapeutically effective amount of a Vitamin A agent.

The Vitamin A agent, according to the present invention, is any analog of Vitamin A, such as, but not limited to, a Vitamin A metabolite, a Vitamin A agonist and a Vitamin A derivative. The Vitamin A agent can further be a prodrug of each of the above agents.

Preferably, his method of the present invention is effected by administering to the subject a therapeutically effective amount of nicotinamide, in combination with all-trans-retinoic acid, which is also referred to herein as “atRA”.

All-trans-retinoic acid is a well-known metabolite of Vitamin A. As this compound is a FDA approved drug, it is widely used in a variety of therapeutic applications, including skin disorders. However, the presently known methods that utilize atRA or other Vitamin A agents, are limited by the skin irritations that are often caused by of these compounds.

As is described in detail in the Examples section that follows, a combination of all-trans-retinoic acid (atRA) and NA was found to be more effective in promoting differentiation and inhibiting proliferation of human epidermal cells, than the sum of the effects of each of these compounds separately. In fact, at the tested concentrations (0.1 nM-10 nM, atRA was found inactive toward proliferation and/or differentiation of epidermal cells. This range of concentrations includes considerably low concentrations and is therefore not expected to cause skin irritations.

Other Vitamin A metabolites, as well as agonists, derivatives and prodrugs of Vitamin A are expected to act similarly when used in the context of the present invention. Also, Vitamin A agents are expected to exert synergistic effect on the proliferation and/or differentiation of epidermal cells, when used in combination with cADPR and its related compounds described hereinabove

Representative examples of other Vitamin A agents that are useful in the context of the present invention include, without limitation, the well-known variety of retinoic acid receptor (RAR) agonists. These include, without limitation, chromans, thiochromans, tetrahydroquinolines, substituted tetrahydronaphthalenes, substituted dihydronaphthalenes, trisubstituted phenyls, aromatic tetracyclic compounds, substituted cyclohexanes, substituted cyclohexenes, substituted cyclohexanedienoic acids, substituted adamentanes, substituted diaryl and heteroaryl compounds and many more. Hence, in a preferred embodiment of the present invention, the Vitamin A agonist comprises a retinoic acid receptor agonist.

For the purpose of convenience, and unless otherwise defined, the term “agent” or “agents” is used hereinafter to describe a NA agent or a cADPR agent, as is defined hereinabove. The phrase “combination of agents” is used hereinafter to describe all the optional combinations of agents that can be used in the context of the present invention, such as, but not limited to, a combination of a NA agent and a cADPR agent, a combination of a NA agent and a Vitamin D3 agent or a Vitamin A agent, a combination of a cADPR agent and a Vitamin D3 agent or a Vitamin A agent, a combination of a NA agent, a cADPR agent and a Vitamin D3 agent or a Vitamin A agent, and more

As is mentioned in brief hereinabove, while reducing the present invention to practice, it was further surprisingly found that long-term (e.g., 6 months) NA-treated human keratinocytes exhibit high resistance to hydrogen peroxide-induced oxidative stress. These findings demonstrate the capability of a nicotinamide agent to act as a strong antioxidant, which increases the anti-oxidative properties of epidermal cells. This feature of a NA agent makes it highly beneficial as, for example, a strong anti-aging agent of skin. This feature further provides a NA agent with the ability to act as an anti-cancer protector of human epidermal cells, as is discussed hereinbelow.

As nicotinamide and cADPR were both found highly active in the treatment of hyperproliferative epidermal pathologies, as is demonstrated herein, it is expected that like nicotinamide, cADPR or any analog thereof, as defined hereinabove, would also exert an anti-oxidative effect on epidermal cells by increasing the anti-oxidative properties of the cells. Moreover, it is expected that all the combinations of agents described and defined hereinabove would exert synergistic anti-oxidative effects.

Hence, according to further aspects of the present invention, there are provided methods of increasing anti-oxidative properties of epidermal cells, preferably human epidermal cells.

These methods of the present invention are effected by contacting the cells with an effective amount of the agents or combinations of agents of the present invention, as described hereinabove.

Hence, in one exemplary particular, a method according to this aspect of the present invention is effected by contacting the cells with a NA agent and/or a cADPR agent.

In another exemplary particular, a method according to this aspect of the present invention is effected by contacting the cells with a NA agent in combination with a Vitamin D3 agent or a Vitamin A agent.

In yet another exemplary particular, a method according to this aspect of the present invention is effected by contacting the cells with a cADPR agent in combination with a Vitamin D3 agent or a Vitamin A agent.

As used in the context of this and the following aspects of the present invention, the phrase “effective amount” and “therapeutically effective amount” describes an amount of the agent that is sufficient to substantially increase the anti-oxidative properties of affected cells and hence, for example, abrogate, substantially inhibit, slow or reverse the progression of oxidative processes in epidermal cells, substantially ameliorate aging and oxidative-induced symptoms or injuries of epidermal cells or substantially prevent the appearance of clinical symptoms associated with aging of epidermal cells.

Preferably, the method according to this aspect of the present invention is effected by contacting the cells with nicotinamide. The effective amount of nicotinamide preferably ranges between about 1 Mm and about 50 mM, more preferably between about 1 mM and about 20 mM and most preferably between about 5 mM and about 15 mM.

Epidermal cells that are treatable by these methods of the present invention include, for example, epidermal cells with symptoms of skin aging (dryness, roughness, burning and atrophy of the skin, itching, cold intolerance, wrinkles, heperpilosity, alopecia), and epidermal cells that are involved in natural or oxidative stress-inducing aging processes. Furthermore, skin cells characterized by increasing sensitivity to oxidative injury, such as cells with predisposition to initiation of tumors can also be treatable by these methods. Increasing the anti-oxidative properties of such cells provides for anti-cancer protection of these cells.

Hence, the methods of increasing anti-oxidative properties of epidermal cells can be efficiently used, for example, in the treatment of aging of epidermal cells or in anti-cancer protection of epidermal cells that are relatively susceptible to the oxidative initiation of cancer tumors. As the agents and the combination of agents described hereinabove were found highly active as antiproliferative agents for treating epidermal hyperproliferative pathologies, and as anti-oxidants which increase the anti-oxidative properties of epidermal cells, according to further aspects of the present invention, there are provided compositions which are identified for use in the treatment of benign or malignant hyperproliferative pathologies and/or for use in the treatment of conditions whereby increasing anti-oxidative properties of epidermal cell is advantageous. These compositions can be either pharmaceutical compositions for therapeutic uses, and/or cosmetic or cosmeceutical compositions.

Hence, according to these aspects of the present invention, there are provided pharmaceutical, cosmetic or cosmeceutical compositions.

These compositions are identified for use in the treatment of benign or malignant hyperproliferative pathologies, as is detailed hereinabove, and/or in the treatment of conditions whereby increasing anti-oxidative properties of epidermal cell is advantageous.

Conditions whereby increasing anti-oxidative properties of epidermal cell is advantageous or, in other words, conditions that require a treatment in which anti-oxidative properties of epidermal cell are increased, include, for example, aging of epidermal cells and cancer.

As used herein, the phrase “aging of epidermal cells” and “aging of the skin” describes all the symptoms associated with physiological aging of the epidermis such as, but not limited to, wrinkles, loss of elasticity, decreased metabolism, dryness, roughness, burning and atrophy of skin, itching, heperpilosity and alopecia.

The treatment of a condition such as aging of the epidermal cells, includes, according to the present invention, treatment of the symptoms described hereinabove with respect to aging of epidermal cells. This treatment further includes prevention of these symptoms, and in particular aging signs, before they occur.

When the condition is cancer, increasing the anti-oxidative properties of epidermal cells is highly advantageous as it provides for an anti-cancer protection of the cells.

As used herein, the phrase “anti-cancer protection” describes a condition in which epidermal cells are characterized by increased sensitivity to oxidative injury, such as cells with predisposition to initiation of tumors, and therefore require anti-cancer protection.

The treatment of such a condition, according to the present invention, includes increasing the anti-oxidative properties of epidermal cells that are in need for anti-cancer protection, as described hereinabove. Since the formation of cancer tumors in epidermal cells typically occurs as a result of oxidative processes, increasing the anti-oxidative properties of epidermal cells, can serve for protecting these cells from processes that lead to cancer.

In one embodiment, the pharmaceutical, cosmetic or cosmeceutical compositions of the present invention comprise, as an active ingredient, a therapeutically effective amount, as this phrase is defined hereinabove, of a nicotinamide agent, as described hereinabove and/or a therapeutically effective amount of a cADPR agent, as described hereinabove, and a pharmaceutically, cosmetically or cosmeceutically acceptable carrier.

Hence, a pharmaceutical, cosmetic or cosmeceutical composition according to this embodiment of the present invention, comprises as an active ingredient, either nicotinamide, agonist, derivative or metabolite thereof or prodrugs thereof cADPR, agonist, derivative or metabolite thereof or prodrugs thereof or a combination thereof.

Optionally and preferably, a pharmaceutical, cosmetic or cosmeceutical composition according to this embodiment of the present invention, comprises a combination of active ingredients and hence comprises both a NA agent and a cADPR agent, which combination exerts synergistic effect on proliferation and differentiation of various epidermal cells, as is described hereinabove.

In a typical pharmaceutical, cosmetic or cosmeceutical composition of the present invention, the therapeutically effective amount of nicotinamide ranges between about 1 mM and about 50 mM. Preferably, it ranges between about 0.5 mM and about 20 Mm. More preferably, it ranges between about 1 mM and about 10 Mm. Most preferably, it ranges between about 2.5 mM and about 5 mM.

The therapeutically effective amount of cADPR, according to the present invention, preferably ranges between about 1 μM and 100 μM, more preferably between about 10 μM and about 50 μM and most preferably between about 25 μM and about 50 μM.

Hence, in pharmaceutical, cosmetic or cosmeceutical compositions that include a combination of NA and cADPR, a preferred concentrations ratio between these agents ranges between 100:1 and 200:1 (by weight). A preferred pharmaceutical, cosmetic or cosmeceutical composition according to the present invention therefore comprises nicotinamide at a final concentration that ranges between about 2.5 mM and about 5 mM and cADPR at a final concentration that ranges between about 25 μM and about 50 μM.

As is discussed hereinabove, when a NA or a cADPR agent was used in combination with either a Vitamin D3 agent or a Vitamin A agent, the resulting combination was found highly effective in modulating proliferation and/or differentiation of epidermal cells.

Hence, in another embodiment of the present invention, the pharmaceutical, cosmetic or cosmeceutical compositions include, as a combination of active ingredients, a therapeutically effective amount of either a NA agent, as described hereinabove, or a cADPR agent, as described hereinabove, in combination with a therapeutically effective amount, as this phrase is defined hereinabove, of a Vitamin A agent or a Vitamin D3 agents, as these agents are described in detail hereinabove.

In one exemplary particular, a pharmaceutical, cosmetic or cosmeceutical composition according to this embodiment of the present invention comprises a therapeutically effective amount of a NA agent, a therapeutically effective amount of a Vitamin D3 agent and a pharmaceutically, cosmetically or cosmeceutically acceptable carrier A preferred pharmaceutical, cosmetic or cosmeceutical composition in this particular comprises therapeutically effective amounts of nicotinamide and a Vitamin D3 metabolite. Preferably, die vitamin D3 metabolite is 1α,25 dihydroxy-vitamin D₃.

A preferred concentration of the NA in this composition is within the ranges defined hereinabove. A preferred ratio between the NA and the Vitamin D3 metabolite ranges between about 5000:1 and about 500,000:1 and preferred final concentrations of the Vitamin D3 metabolite typically ranges between 1 nM and 10000 nM, and more preferably between about 0.01 μM (10 nM) and about 1 μM (1000 nM).

In another exemplary particular, a pharmaceutical, cosmetic or cosmeceutical composition according to this embodiment of the present invention comprises a therapeutically effective amount of a NA agent, a therapeutically effective amount of a Vitamin A agent, as these phrases are defined hereinabove, and a pharmaceutically, cosmetically or cosmeceutically acceptable carrier. A preferred pharmaceutical, cosmetic or cosmeceutical composition of this particular comprises therapeutically effective amounts of nicotinamide and a Vitamin A metabolite. Preferably, the vitamin A metabolite is all-trans-retinoic acid.

A preferred concentration of the NA in this composition is within the ranges defined hereinabove. A preferred ratio between the NA and the Vitamin A metabolite ranges between about 5×10⁵:1 and about 25×10⁷:1. Preferred final concentrations of the Vitamin A metabolite typically ranges between about 0.1 nM and about 100 nM, and more preferably between about 1 nM and about 10 nM.

All the pharmaceutical, cosmetic or cosmeceutical compositions of the present invention include a pharmaceutically, cosmetically or cosmeceutically acceptable carrier.

As used herein, the phrases “cosmetically acceptable carrier” and cosmeceutically acceptable carrier” refer to a carrier or a diluent that does not cause significant irritation to the skin and does not abrogate tie biological activity and properties of the applied active agent.

Examples of cosmetically or cosmeceutically acceptable carriers that are useful in the context of the present invention include, without limitation, emulsions, creams, aqueous solutions, oils, ointments, pastes, gels, lotions, milks, foams, suspensions and powders.

The cosmetically or cosmeceutically acceptable carrier of the present invention may include, for example, a thickener, an emollient, an emulsifier, a humectant, a surfactant, a suspending agent, a film forming agent, a foam building agent, a preservative, an antifoaming agent, a fragrance, a lower monoalcoholic polyol, a high boiling point solvent, a propellant, a colorant, a pigment or mixtures thereof

Therefore, the final cosmetic or cosmeceutical composition of the present invention may be, for example, in the form of an oil, a gel, a solid stick, a lotion, a cream, a milk, an aerosol, a spray, an ointment or a fatty ointment and a powder. The cosmetic and cosmeceutical compositions of the present invention are preferably topically applied on the treated epidermal cells.

As used herein, the phrase “pharmaceutically acceptable carrier”, which is also referred to herein interchangeably as “physiologically acceptable carrier” describes a carrier, an excipient or a diluent that does not cause significant irritation to a subject, and particularly to the skin of a subject. Hence, preferred carriers in the pharmaceutical compositions of the present invention are also dermatological acceptable carriers. The carrier does not abrogate the biological activity and properties of the administered compound or combination of compounds. The carrier is typically added to facilitate the administration of the active ingredient(s).

Herein, the term “excipient” describes an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drug agents may be found in “Remington's Pharmaceutical Sciences,” Mack Publishing Co., Easton, Pa., latest edition, which is incorporated herein by reference.

Suitable routes of administration may, for example, include oral, rectal, transmucosal, intestinal or parenteral delivery, including intramuscular, subcutaneous and intramedullary injections as well as intrathecal, direct intraventricular, intravenous, intraperitoneal, intranasal, or intraocular injections.

Preferably, the pharmaceutical compositions of the present invention are administered either topically or subcutaneously.

Pharmaceutical compositions for topical administration are preferably in the form of cream, gel, solution, salve, lotion, ointment or fatty ointment.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

For injection, the compounds (agents) of the invention may be formulated in aqueous solutions, preferably in physiologically compatible buffers such as Hank's solution. Ringer's solution, or physiological saline buffer. For transmucosal administration, penetrants appropriate to the barrier to be permeated are used in the formulation. Such penetrants are generally known in the art.

For oral administration, the agents of the present invention can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, scaled capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the agents for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compositions described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for, parenteral administration include aqueous solutions of the active preparation in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile, pyrogen-free water, before use.

The compound(s) of the present invention may also be formulated in rectal compositions such as suppositories or retention enemas, using, e.g., conventional suppository bases such as cocoa butter or other glycerides.

In addition to the formulations described previously, a composition of the present invention may also be formulated for local administration, such as a depot preparation Such long acting formulations may be administered by implantation (for example subcutaneously or intramuscularly) or by intramuscular injection. Thus, for example, the composition may be formulated with suitable polymeric or hydrophobic materials (for example, as an emulsion in an acceptable oil) or ion exchange resins, or as sparingly soluble derivatives such as sparingly soluble salts. Compositions for topical administration may include, but are not limited to, lotions, suspensions, ointments gels, creams, drops, liquids, sprays emulsions and powders, as is described hereinabove.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Many of the agents in the claimed compositions of the present invention may be provided as physiologically acceptable salts wherein the agent may form the negatively or the positively charged species. Examples of salts in which the agent forms the positively charged moiety include, without limitation, quaternary ammonium (defined elsewhere herein), salts such as the hydrochloride, sulfate, carbonate, lactate, tartrate, maleate, succinate, etc, wherein the nitrogen of the quaternary ammonium group is a nitrogen of a compound of the present invention which reacts with an appropriate acid. Salts in which the agent forms the negatively charged species include, without limitation, the sodium, potassium, calcium and magnesium salts formed by the reaction of a carboxylic acid group in the molecule with the appropriate base (e.g., sodium hydroxide (NaOH), potassium hydroxide (KOH), calcium hydroxide (Ca(OH)₂), etc.).

Pharmaceutical compositions suitable for use in context of the present invention include compositions wherein the active ingredients are contained in an amount effective to achieve the intended purpose, as is discussed and defined hereinabove.

Determination of a therapeutically effective amount is well within the capability of those skilled in the art, especially in light of the detailed disclosure provided herein.

For any agent or combination of agents used within the scope of the invention, the therapeutically effective amount or dose can be estimated initially from cell culture assays. For example, a dose can be formulated in animal models to achieve a circulating concentration range that includes the IC₅₀ as determined in cell culture (e.g., the concentration of the test compound, which achieves a half-maximal inhibition of the epidermal cells proliferation). Such information can be used to more accurately determine useful doses in humans.

Toxicity and therapeutic efficacy of the agents described herein can be determined by standard pharmaceutical procedures in cell cultures or experimental animals, e.g., by determining the IC₅₀ and the LD₅₀ (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma levels of the active moiety which are sufficient to maintain the anti-proliferative effects, termed the minimal effective concentration (MEC). The MEC will vary for each preparation, but can be estimated from in vitro data; e.g., the concentration necessary to achieve 50-90% inhibition of a kinase may be ascertained using the assays described herein. Dosages necessary to achieve the MEC will depend on individual characteristics and route of administration.

Dosage intervals can also be determined using the MEC value. Preparations should be administered using a regimen, which maintains plasma levels above the MEC for 10-90% of the time, preferable between 30-90% and most preferably 50-90%.

It is noted that, in the case of local administration or selective uptake, the effective local concentration of the drug may not be related to plasma concentration. In such cases, other procedures known in the art can be employed to determine the effective local concentration.

Depending on the severity and responsiveness of the condition to be treated, dosing can also be a single administration of a slow release composition described hereinabove, with course of treatment lasting from several days to several weeks or until cure is effected or diminution of the disease state is achieved.

The amount of an agent or a combination of agents to be administered will, of course, be dependent on the subject being treated, the severity of the affliction, the manner of administration, the judgment of the prescribing physician, etc.

The pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Compositions comprising the agents of the invention formulated in a compatible pharmaceutical carrier may also be prepared, placed in an appropriate container, and labeled for treatment of a benign and/or a malignant hyperproliferative epidermal pathology.

Optionally, compositions comprising the agents of the invention formulated in a compatible pharmaceutical, cosmetic or cosmeceutical carrier may also be prepared, placed in an appropriate container, and labeled for treatment of a condition whereby increasing anti-oxidative properties of epidermal cells is advantageous.

Hence, according to further aspects of the present invention, there are provided pharmaceutical, cosmetic and cosmeceutical kits. The pharmaceutical, cosmetic and cosmeceutical kits of the present invention comprise any of the agents or combinations of agents described hereinabove. Whenever a combination of agents is present in the kits of the present invention, the agents are individually packaged in the kit.

The pharmaceutical, cosmetic and cosmeceutical kits are identified, in print, for use in the treatment of a benign and/or a malignant hyperproliferative epidermal pathologies and/or in the treatment of a condition whereby increasing anti-oxidative properties of epidermal cells is advantageous.

As is discussed hereinabove and is further demonstrated in the Examples section that follows, the high efficacy of the agents or the combinations of agents of the present invention in treating hyperproliferative epidermal pathologies is mainly attributed to the high capability of these agents or combination of agents to inhibit the proliferation of epidermal cells.

Hence, according to additional aspects of the present invention there are provided methods of inhibiting proliferation of benign or malignant hyperproliferative epidermal cells.

These methods of the present invention are effected by contacting the cells with a therapeutically effective amount, as this phrase is defined hereinabove, of the agents or combinations of agents of the present invention, as described hereinabove.

In all the methods of this aspect of the present invention, tie cells may be malignant epidermal cells such as, but not limited to, cells from squamous cell carcinoma (SCC) basal cell carcinoma (BCC) or other non-melanoma skin cancers (NMSCs) or alternatively may be hyperproliferative benign cells, such as human keratinocytes from psoriatic skin, and keratinocytes from keratoacanthoma, common warts or seborrhoic keratoses lesions. The cells may also be from other benign skin disorders such as ichthyosis.

Hence, in one exemplary particular, a method according to this aspect of the present invention is effected by contacting the cells with a NA agent and/or a cADPR agent.

In another exemplary particular, a method according to this aspect of the present invention is effected by contacting the cells with a NA agent in combination with a Vitamin D3 agent or a Vitamin A agent.

In yet another exemplary particular, a method according to this aspect of the present invention is effected by contacting the cells with a cADPR agent in combination with a Vitamin D3 agent or a Vitamin A agent.

Additional objects, advantages, and novel features of the present invention will become apparent to one ordinarily skilled in the art upon examination of the following examples, which are not intended to be limiting. Additionally, each of, the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### MATERIALS AND EXPERIMENTAL METHODS

Cell Cultures:

The immortalized human keratinocyte HaCat cells were routinely cultured in 75 cm² flasks using Eagle's minimal essential medium (MED-EAGLE) supplemented with 5% fetal calf serum (FCS) and 1% antibiotics (penicillin 20 units/ml; streptomycin 20 μg/ml and mystatin 2.5 units/ml) at 37° C. in 95% air/5% CO₂. The medium was replaced every 3-4 days.

Long-term cultures of HaCat cells wilt NA were obtained by cultivating HaCat cells, for 6 months, in routinely used medium, supplemented with 10 mM NA or 20 mM NA.

Other long-term cultures of cells with other agents are similarly obtained by cultivating HaCat cells, for a prolonged period of time, in routinely used medium supplemented with combinations of NA and cADPR, 1α,25-dihyroxy-vitamin D3 and/or atRA.

Human Epidermal Keratinocytes (passages 3-6), obtained from normal face-lift surgery, were cultivated in serum-free KGM®-2 BulletKit® CC-3107 (Clonetics, USA) medium with low calcium for accelerated proliferation of the keratinocytes.

Epidermal carcinoma A431 cells were cultured in Dulbecco's modified Eagle's medium (DMEM) supplemented with 10% FCS and antibiotics as above.

Reagents;

Nicotinamide (NA); cyclic adenosine diphosphate-ribose (cADPR); calcitriol (1α,25-dihyroxy-vitamin D3); all trans retinoic acid (atRA; Vitamin A acid; Tretinoin); 3-(4,5-dimethylthiazo-2-yl)-2,5-diphenyltetrazolium bromide (MTT), propidium iodide; dimethylsulphoxide (DMSO); bovine serum albumin (BSA); sucrose; trisodium citrate; igepal CA-630 (NP-40); Tris-(hydroxymethyl)aminomethane; trypsin; trypsin inhibitor; ribonuclease A; spermintetrahydrochloride; sodium dodecylsulfate (SDS); β-mercaptoethanol and hydrogen peroxide (H₂O₂), were all obtained from Sigma (USA).

Eagle's minimal essential medium (MEeM-EAGLE); DMEM; antibiotics; fetal calf serum (FCS); L-glutamine; Dulbecco's phosphate buffered saline (PBS); and trypsin 0.05%-EDTA solution were obtained from Biological Industries (Israel).

Keratinocyte Growth Medium®-2 Bullet Kit® CC-3107 (for accelerated proliferation) was received from BioWhittaker, Inc. A Cambrex Company, Clonetics, USA).

Anti-human cytokeratin 10 (NCL-CK10) and involucrin (NCL-INV) mouse monoclonal antibodies were obtained from Novocastra Laboratories Ltd. (UK) and Cy™ 2-conjugated goat anti-mouse IgG was obtained from Jackson Immunoresearch Laboratories, Inc. (USA).

HaCat and A431 cells were propagated in 25 cm² or 75 cm² tissue culture flasks (Corning, USA) and 24-well and 96-well tissue culture plates (Corning, USA) were used for incubation of the cells with different doses of NA (1-50 mM/I), cADPR (1-50 μM) vitamin D3 (1-10000 nM) and atRA (0.1-10000 nM).

Proliferation Assays (MTT Method):

The viability and/or proliferation of HaCat and A431 cells and Cultured Human Epidermal Keratinocytes, following treatment with various concentrations of nicotinamide (NA) and/or various concentrations of cADPR, Vitamin D3 metabolites and Vitamin A metabolites, were determined by the MTT assay, according to the procedure described in Mosmann, T: Rapid colorimetric assay for cellular growth and survival: Application to proliferation and cytotoxicity assays, J. Immunol. Meth., 65: 55-63, (1983), in 96-well microtiter plates.

In brief, an equal number of cells were seeded in each welt and incubated for 24 hours. NA or a combination of NA and another agent (NA and cADPR; NA and vim D3 metabolite; NA with Vitamin A metabolite), in various concentrations, was added thereafter and the wells were incubated for additional 72 hours. Twenty microliters (20 μ) of 5 mg/ml MTT in phosphate buffered saline (PBS) without Ca⁺² and Mg⁺² were then added to each well. The plates were placed in an incubator with CO₂ and MTT was converted to the insoluble MTT-formazan crystals by mitochondrial dehydrogenases during 3.5 hours. The medium was then removed and the obtained formazan crystals were dissolved in 0.2 ml of DMSO. The amount of formazan was quantified in an ELISA-reader at 550 nm. Background values at 650 nm were subtracted. The presented Data are results from three independent experiments.

Differentiation Assays:

Cornified Envelope Formation:

Late differentiation processes in HaCat cells treated with nicotinamide were measured by determining the Cornified cell envelope formation, according to the procedure described in Sun T-T, Green, H: Differentiation of the epidermal keratinocytes in cell culture: formation of cornified envelope, Cell, 9: 511-521, 1976.

In brief, cells were seeded in 24-well tissue culture plates and after attachment (24 hours) were exposed to various concentrations of NA (0, 4, 10, 15 and 20 mM) for 96 hours. The cells were thereafter detached and re-suspended in medium. Counting of total and basal (small, rounded) cells was performed using hemocytometer in tetraplicate aliquots. The remaining cells were spun down, treated with 10 mM Tris-HCl (pH 7.4) supplemented with 1% β-mercaptoethanol and 1% SDS for 10 minutes and cornified envelope cells were counted in tetraplicate aliquots using hemocytometer. The presented data were results of three independent experiments.

Indirect Immunofluorescence:

Effects of NA on early (keratin k10 expression) and late (involucrin expression) differentiation processes in HaCat cells were estimated by indirect immunofluorescence.

In brief, 2×10⁴ cells/ml were seeded on glass coverslips into Petri dishes with 0, 5, 10 and 20 mM NA. After 72 hours of incubation, cells on the glass coverslips were washed with PBS, fixed by ice-cold mixture of methanol: acetone (1:1) and incubated at −20° C. for 10 minutes. Fixed cells were thereafter washed in PBS and incubated with blocking buffer (1% BSA in PBS) for 10 minutes, to minimize non-specific absorption of the primary antibodies to the coverslips. Thereafter, the cells were incubated for 1 hour with monoclonal antibodies (Keratin 10 expression was detected by antihuman mouse monoclonal antibody, at 1/50 final dilution; Involucrin expression was detected by antihuman involucrin mouse monoclonal antibody at 1/100 final dilution), at 37° C. hour in a humidified chamber. Exhaustive, PBS-washed cells were incubated with Cy™ 2-conjugated goat anti-mouse IgG, at 1/50 final dilution, for 30 minutes at room temperature. The obtained slides were viewed under Zeiss microscope (Axioskop-2) equipped with epifluorescence optics and the appropriate filters to avoid cross-channel contamination. The level of keratin 10 and involucrin expression was estimated by counting the positive cells relative to the total cell number. In each slide, at least 500-1000 cells were scored. The presented data is a mean of three independent experiments.

DNA Labeling and Flow Cytometry Analysis:

HaCat and A431 cells were seeded in 25 cm² tissue culture flasks and incubated for 72 hours with 0, 5, 10 and 20 mM of NA. Cells treated with 5% ethanol served as positive control of apoptosis. The nuclei for flow cytometry analysis of DNA were prepared by a detergent trypsin method with propidium iodide, according to the procedure described in Lars L Rindelov: A detergent trypsin method for the preparation of nuclei for FACS DNA analysis, Cytometry 3(5)323-327, 1983.

In brief, the cells (10⁶ per tube) were washed with PBS. The cell pellet was re-suspended in 40 μl citrate buffer (pH 7.6) supplemented with 250 mM sucrose, 40 mM trisodium citrate and 5% DMSO. The re-suspended cells were then incubated in 450 μl solution of trypsin (0.15 mg/ml, pH 7.6) for 10 minutes, and thereafter with trypsin inhibitor and ribonuclease A for another 10 minutes. A hundred (100) μg/ml of fluorochrome solution containing propidium iodide were then added to nuclei, The tubes were placed in the dark and the flow cytometry analysis was carried out in fluorescence-activated cell sorter (FACScan; Becton Dickinson, Calif.). The level of apoptosis was determined using the Cell Quest Program of Becton Dickonson. Each experiment was repeated three times.

Statistical Analysis:

Results are presented as mean±standard deviation of the mean (mean±SD). Statistical significance (P<0.05) was derived by Student's t-test.

### EXPERIMENTAL RESULTS

NA Effect on Epidermal Cell Proliferation:

2×10⁴ cells/Ml of immortalized human keratinocyte IIaCat cells, Cultured Human Epidermal Keratinocytes and 5×10³ cells/ml of squamous carcinoma A431 cells were incubated with varying amounts of NA for a period of 72 hours. The cells proliferation was estimated by the MTT method, described hereinabove, and was expressed as the percent from control (untreated cells).

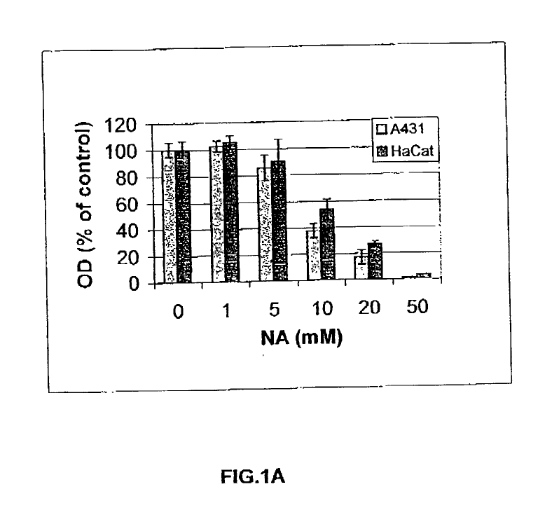

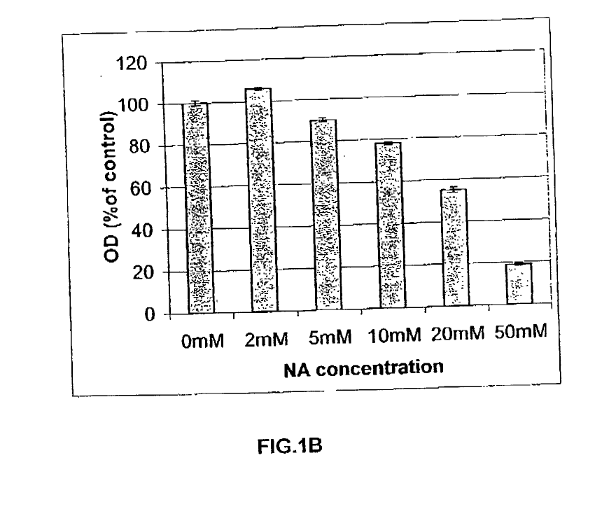

The results are presented in FIGS. 1a and 1b and indicate that NA significantly inhibited the cell proliferation of all the tested cells.

Effect of cADPR on Cell Proliferation:

HaCat cells and A431 cells, as described hereinabove, were incubated with varying amounts of cADPR for 72 hours.

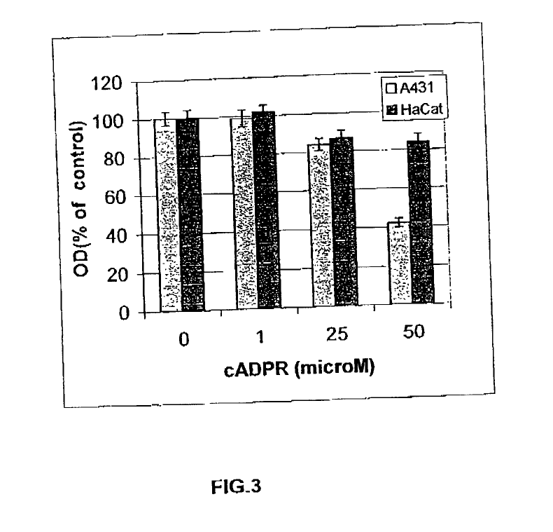

As shown in FIG. 3, cADPR, at concentrations of 25 and 50 μM, exerted effective inhibition of cell proliferation.

Effect of Vitamin D3 Metabolite and Vitamin A Metabolite on Cell Proliferation:

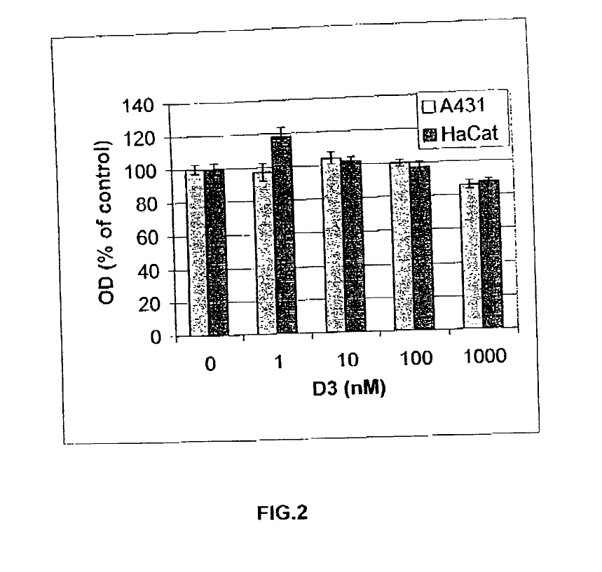

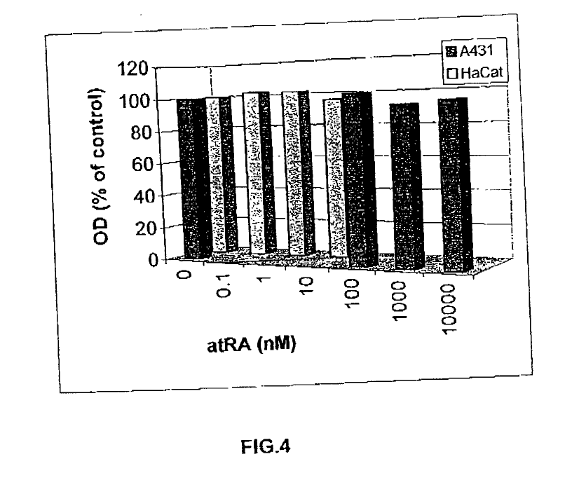

HaCat cells and A431 cells, as described hereinabove, were incubated with varying amounts of Vitamin D3 metabolite (1α25(OH)₂D3) or Vitamin A metabolite (atRA) for 72 hours. The obtained results are presented in FIG. 2 and FIG. 4, respectively.

As shown in FIGS. 2 and 4, neither Vitamin D3 metabolite nor Vitamin A metabolite, alone, at the tested concentrations, affected the proliferation of HaCat and A431 cell lines.

Synergistic Effects of NA and a Vitamin D3 Metabolite on Cell Proliferation:

HaCat cells and A431 cells were incubated with the Vitamin D3 metabolite 1α25(OH)₂D3 alone and with a combination of 1α25(OH)₂D3 and NA, for a period of 72 hours.

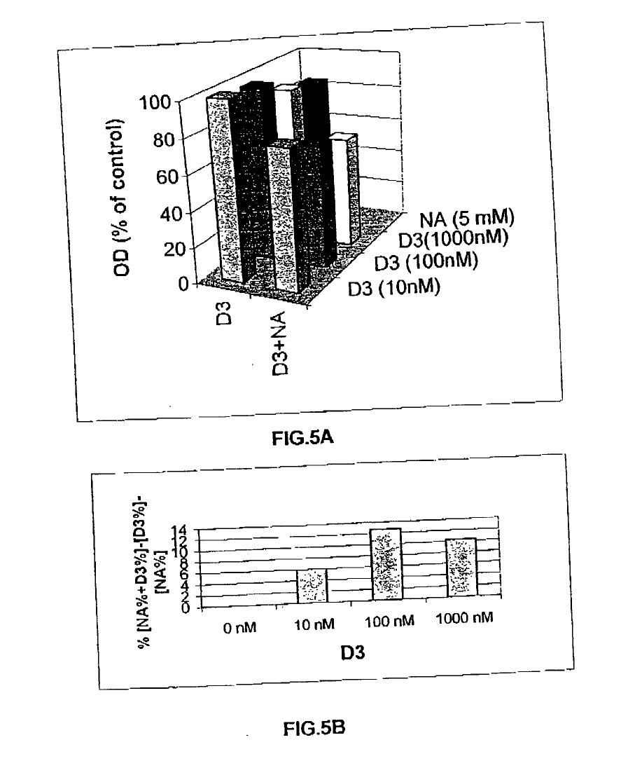

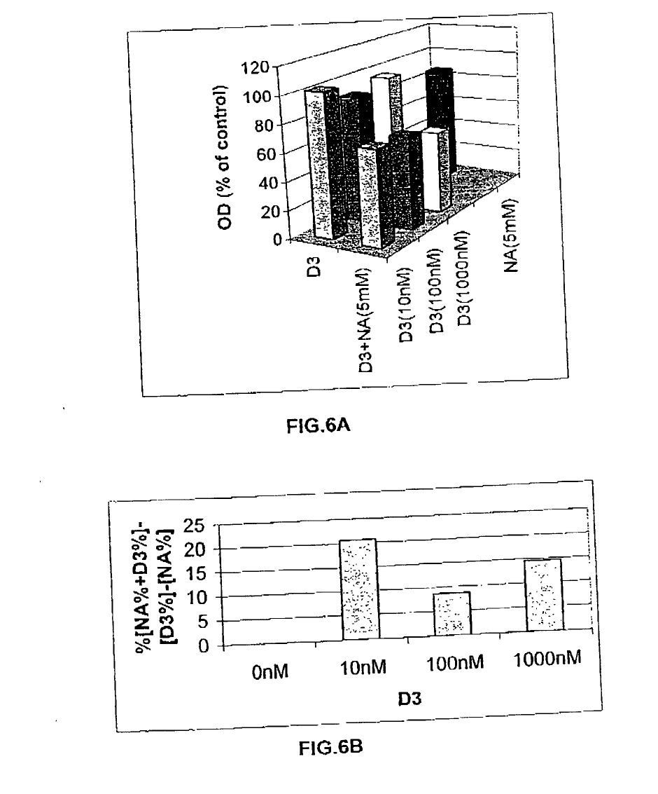

FIGS. 5a and 6a present the results obtained with the above combination in HaCat and A431 cell lines, respectively.

FIGS. 5b and 6b present a deduction of the anti-proliferative effects of 1α25(OH)₂D3 and NA, when applied separately on the cell lines as described hereinabove, from the anti-proliferative effect of the combination of NA and 1α25(OH)₂D3, presented in FIGS. 5a and 6a. FIG. 5b presents the deduction results in HaCat cells and FIG. 6b presents the deduction results in A431 cells.

As is shown in FIGS. 5b and 6b, the anti-proliferative effect of the combination of NA and 1α25(OH)₂D3 is substantially higher than the summation of the anti-proliferative effects of each of these compounds separately. When a combination of 100 nM 1α25(OH),D3 and 5 mM NA was used in HaCat cells, enhancement of 12% was observed in the inhibition of cell proliferation. When a combination of 10 nM of 1α25(OH)₂D3 and 5 mM NA was used in A431 cells, enhancement of 20% was observed in the inhibition of cell proliferation. These results clearly demonstrate the synergistic effect of a combination of NA and a Vitamin D3 metabolite in inhibiting epidermal cell proliferation.

Synergistic Effect of cADPR and NA on Cell Proliferation:

2×10⁴ cells/ml HaCat cells and 5×10³ cells/ml squamous carcinoma A431 cells were incubated with NA, at a concentration of 5 mM and 2.5 mM, respectively, and with varying amounts of cADPR (25-50 μm).

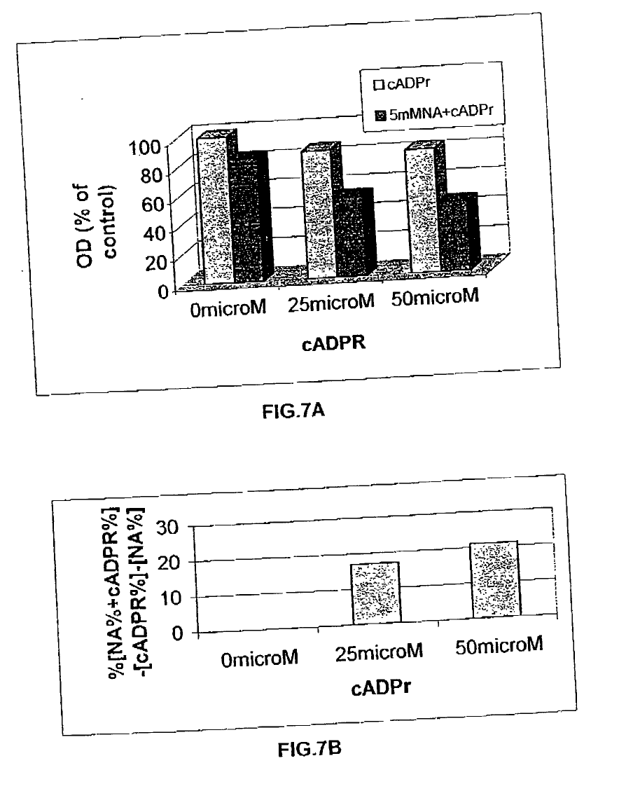

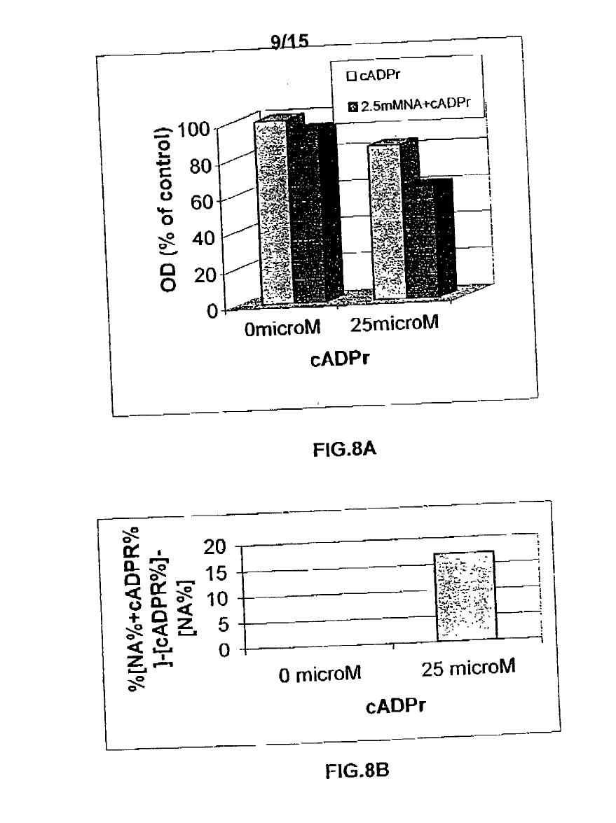

FIGS. 7a and 8a present the results obtained in HaCat cells and in A431 is cells, respectively. FIGS. 7b and 8b present the respective deduction of the anti-proliferative effects of cADPR and NA, when applied separately on each of the cell lines as described hereinabove, from the anti-proliferative effect of the combination, presented in FIGS. 5a and 6a.

As is shown in FIGS. 7a and 7b, a combination of 50 μM cADPR and 5 mM NA exerted a synergistic effect of more than 20% in inhibiting proliferation as compared with the effect of each of these agents alone, at the same concentrations, in HaCat cells. As is shown in FIGS. 8a and 8b, a combination of 25 μM cADPR and 2.5 mM NA exerted a synergism of above 16% in inhibiting proliferation of A431 cells, as compared with the inhibition of each of these components alone.

Synergistic Effect of a Combination of NA and Vitamin A Metabolite on Proliferation of Epidermal Cells:

HaCat cells and A431 cells were incubated with 5 mM NA and 2.5 mM NA, respectively, and with varying concentrations of atRA (0.1 nM-1 μM).

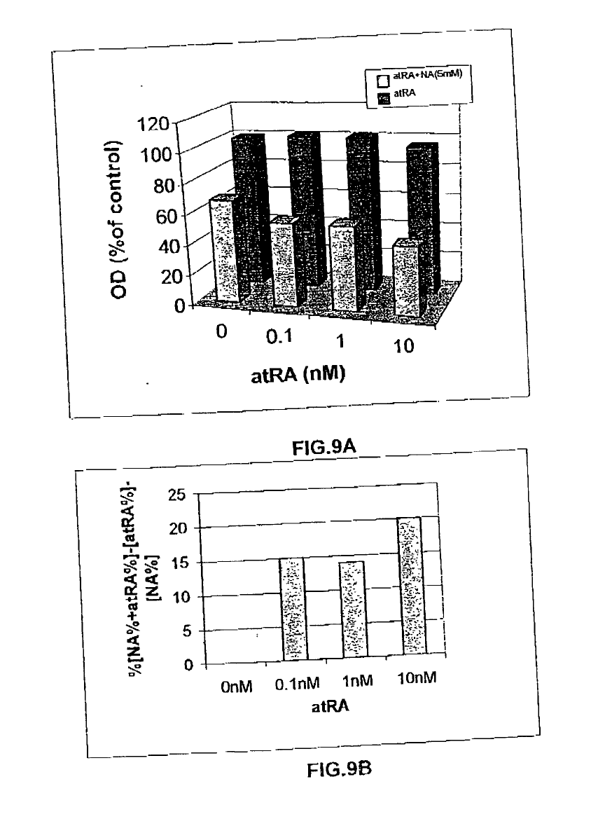

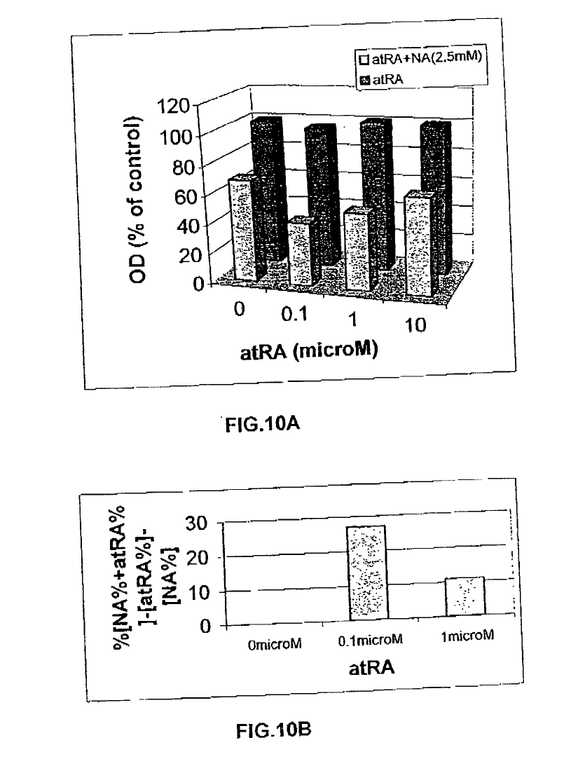

As is detailed hereinabove, FIGS. 9a and 10a present the anti-proliferative effect of this combination in HaCat cells and in A431 cells, respectively, while FIGS. 9b and 10b demonstrate the respective synergistic effect of this combination in theses cell lines.

As is shown in FIGS. 9b and 10b, a synergistic anti-proliferative effect of more than 25% was observed in A431 cells with a combination that included a concentration of 0.1 μM atRA (FIG. 10b) and a synergism of more than 20% was observed in HaCat cells with a combination that included a concentration of 10 μM of atRA (FIG. 9b).

Effect of NA on Cell Differentiation and Apoptosis:

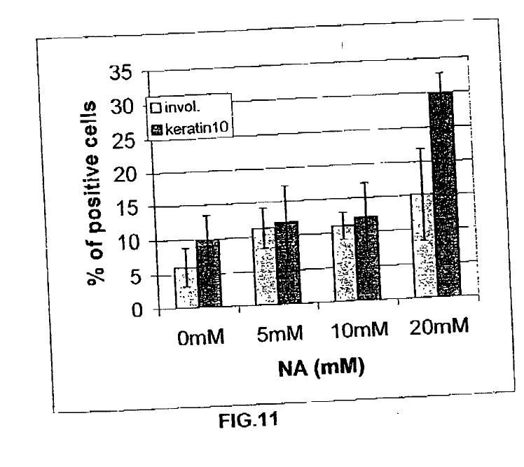

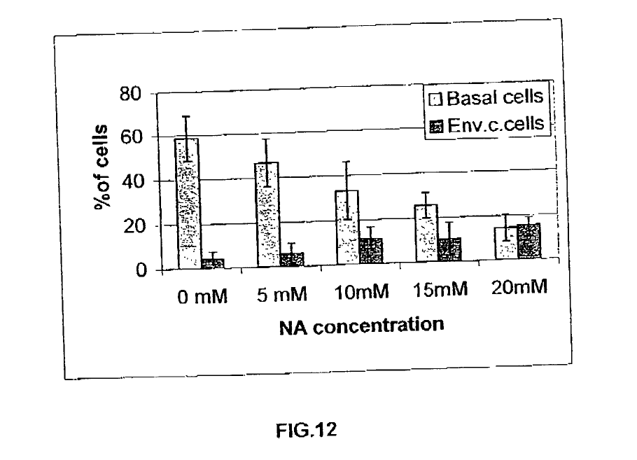

The effect of NA on differentiation was determined by indirect immunofluorescence of keratin K10 and involucrin and by cornified envelope formation, as described above, and the results are presented in FIG. 11 and FIG. 12, respectively.

As is shown in FIG. 11, the NA treatment simulated both expressions of keratin 10 (K10) and involucrin, which are markers of early and late differentiation processes of the epidermal cells, respectively.

As is shown in FIG. 12, the NA treatment also affected the ratio between the amount of cells and envelope cornified cells. A higher proportion of enveloped cornified cells, which are more differentiated cells, in the tested cells was observed.

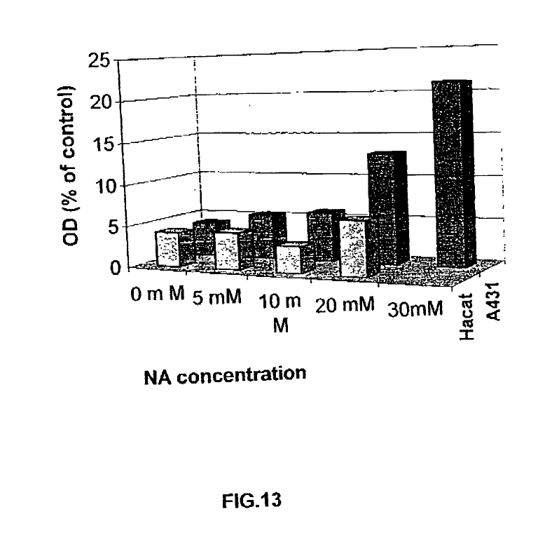

The effect of NA on the level of apoptosis was also determined in HaCat and A431 cells. As is shown in FIG. 13, the determined apoptosis levels show that NA becomes cytotoxic to the cells at a concentration of 30 mM in A431 cells and at a concentration of 50 mM in HaCat cells. These results are significant since they demonstrate that the effect of NA on cell proliferation, as is expressed, for example, in FIG. 1, is effectively exerted by NA concentrations that are lower than the cytotoxic level of NA, namely, at concentrations lower than the concentrations that are toxic to cells.

Resistance of HaCat Cells Long-Term Cultured With NA and/or cADPR, With or Without Vitamin D3 or A Metabolites, to Hydrogen Peroxide-Induced Oxidative Stress:

Long-term cultured cells with various agents or combination of agents are prepared as described hereinabove and are thereafter incubated with increasing concentrations of hydrogen peroxide for 24 hours. The cytotoxicity is estimated by the MTT method described above.

Resistance of HaCat Cells Long-Term Cultured with NA (10 mM) to Hydrogen Peroxide-Induced Oxidative Stress:

2×10⁴ cells/ml immortalized human keratinocyte HaCat cells cultivated routinely, or the same amount of HaCat cells cultured with 10 mM NA during 6 months, as is described hereinabove, were incubated with increasing concentrations of hydrogen peroxide for 24 hours. The cytotoxicity was estimated by the MTT method described above and was expressed as the percent from control (untreated cells).

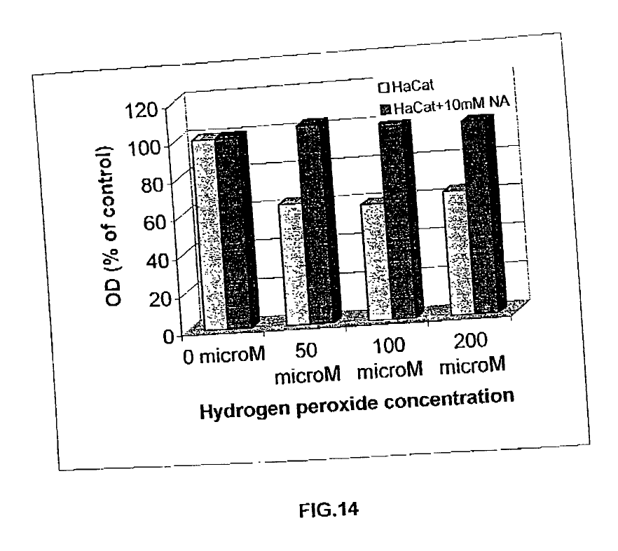

The obtained results are presented in FIG. 14, which demonstrates that while HaCat cells that were cultivated routinely (without NA supplementation) were significantly injured by hydrogen peroxide, the HaCat cells long-term cultured with NA remained unaffected. These data indicate that long-term treatment with NA increases the anti-oxidative properties of human epidermal cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED BY NUMERALS

1. Otonkoski T., Beatie, G. M., Mally, M. I., Ricordi, S., Hayek, A., “Nicotinamide is a potent inducer of endocrine differentiation in cultured human fetal pancreatic cells”, J. Clin. Invest., 92:1459-1466, (1993).

2. Yamada, K., Nonaka, K., Hanafusa, T., Miyazaki, A., Toyoshima, H., Tarui, S: “Preventive and therapeutic effects of large-dose nicotinamide injections on diabetes associated with insulitis: an observation in nonobese diabetic (NOD) mice”, Diabetes, 31:749-753, (1982).

3. Elliot, R. B., Chase, H. P.: “Prevention or delay of Type 1 (insulin-independent) diabetes mellitus in children using nicotinamide”. Diabetologia, 34:362-365, (1991).

4. Petley, A., Macklin, B., Renwick, A. G., Wilkin, T. J.: “The Pharmacokinetics of Nicotinamide in Human and Rodents”. Diabetes, 44:152-155,(1995).

5. Grant, R. S., Kapoor, V.: “Murine glial cells regenerate NAD, after peroxide-induced depletion, using either nicotinic acid, nicotinamide, or quinolinic acid as substrates”, J. Neurochem., 70:1759-1763, (1998),

6. Morimoto S., Yoshikawa, K., Konzuka, T., et al., “An open study of vitamin D3 treatment in psoriasis vulgaris”, Br. J. Dermatol., 115:421-429, (1986).

7. Lee H. C., Specific binding, of cyclic ADP-ribose to calcium-storing microsomes from sea urchin eggs. J. Biol. Chem., 266:2276-2281 (1991).

8. Beers K. W., Chini, E. N., and Dousa, T. P.: “All-trans-retinoic acid stimulates synthesis of cyclic ADP-ribose in renal LLC-PK₁”, J. Clin. Invest., 95:2385-2390, (1995).

9. Ockenfels, H. M., Nuβbaum, G., Schultewolter, T., Burger, P. M., Goos, M.: “Cyclosporin A, FK506 and dithranol alter tyrosine-specific protein phosphorylation in HaCat keratinocytes”. Arch. Dermatol. Res., 287:304-309, (1995).

10. Paramio, J. M., and Jorcano, J. L.: “Role of protein kinases in the in vitro differentiation of human HaCat cells” Brit. J. Dermatol., 137:44-50, (1997)).

11. Ahmad, N., Feyes, D. K., Agarwal, R., Mukhtar, H: Photodynamic therapy results in induction of WAF1/CIPI/P21 leading to cell cycle arrest and apoptosis. Proc. Natl. Acad. Sci. USA, 95:6977-6982, (1998).

12. Nikoloff, B. J., Fisher, G. J., Mitra, R. S., Voorhees, J. J.: “Additive and Synergistic Antiproliferative Effect of Cyclosporin A and Gamma Interferon on Cultured Human Keratinocytes” Amer. J. Pharmacol., 131:12-18, (1988).

13. Sun T-T, Green, H: Differentiation of the epidermal keratinocyte in cell culture: formation of cornified envelope, Cell, 9:511-521, (1976).

14. Mosmann, T: Rapid colorimetric assay for cellular growth and survival: Application to proliferation and cytotoxicity assays. J. Immunol. Meth., 65:55-63, (1983).

15. Sun T-T, Green, H: Differentiation of the epidermal keratinocyte in cell culture: formation of cornified envelope Cell, 9:511-521 (1976).

What is claimed is:

1. A method of treating a benign or malignant hyperproliferative epidermal pathology in a subject in need thereof, the method comprising:
administering to said subject a therapeutically effective amount of an agent selected from the group consisting of nicotinamide, a nicotinamide agonist, a nicotinamide derivative, a nicotinamide metabolite and prodrugs thereof.

2. The method of claim 1, wherein said hyperproliferative benign epidermal pathology is selected from the group consisting of psoriasis, ichythyiosis, common warts, keratoacanthoma, seborrhoic keratosis and seborrhea.

3. The method of claim 1, wherein said hyperproliferative malignant epidermal pathology is selected from the group consisting of squamous-cell carcinoma (SCC), basal cell carcinoma (BCC) and a non-melanoma skin cancer NMSC).

4. The method of claim 1, further comprising:
administering to said subject, in combination with said agent, a therapeutically effective amount of an agent selected from the group consisting of cyclic adenosine diphosphate-ribose (cADPR), a cADPR derivative, a cADPR metabolite, a cADPR agonist and prodrugs thereof.

5. The method of claim 1, further comprising:
administering to said subject, in combination with said agent, a therapeutically effective amount of an agent selected from the group consisting of Vitamin D3, a Vitamin D3 metabolite, a Vitamin D3 agonist, a Vitamin D3 derivative and prodrugs thereof.

6. The method of claim 5, wherein said Vitamin D3 metabolite is 1α,25-dihydroxy-vitamin D3.

7. The method of claim 1, further comprising:
administering to said subject, in combination with said agent, a therapeutically effective amount of an agent selected from the group consisting of Vitamin A, a Vitamin A metabolite, a Vitamin A agonist, a Vitamin A derivative and prodrugs thereof.

8. The method of claim 7, wherein said Vitamin A metabolite is an all-trans-retinoic acid.

9. The method of claim 7, wherein said Vitamin A agonist is a retinoic acid receptor agonist.

10. A method of treating a benign or malignant hyperproliferative epidermal pathology in a subject in need thereof, the method comprising:
administering to said subject a therapeutically effective amount of an agent selected from the group consisting of cyclic adenosine diphosphate-ribose (cADPR), a cADPR derivative, a cADPR metabolite, a cADPR agonist and prodrugs thereof.

11. The method of claim 10, wherein said hyperproliferative benign epidermal pathology is selected from the group consisting of psoriasis, ichythyiosis, common warts, keratoacanthoma, seborrhoic keratosis and seborrhea.

12. The method of claim 10, wherein said hyperproliferative malignant epidermal pathology is selected from the group consisting of squamous-cell carcinoma (SCC), basal cell carcinoma (BCC) and a non-melanoma skin cancer (NMSC).

13. The method of claim 10, further comprising:
administering to said subject, in combination with said agent, a therapeutically effective amount of an agent selected from the group consisting of nicotinamide, a nicotinamide agonist, a nicotinamide derivative, a nicotinamide metabolite and prodrugs thereof.

14. The method of claim 10, further comprising:
administering to said subject, in combination with said agent, a therapeutically effective amount of an agent selected from the group consisting of Vitamin D3, a Vitamin D3 metabolite, a Vitamin D3 agonist, a Vitamin D3 derivative and prodrugs thereof.

15. The method of claim 14, wherein said Vitamin D3 metabolite is 1α,25-dihydroxy-vitamin D3.

16. The method of claim 10, further comprising;
administering to said subject, in combination with said agent, a therapeutically effective amount of an agent selected from the group consisting of Vitamin A, a Vitamin A metabolite, a Vitamin A agonist, a Vitamin A derivative and prodrugs thereof.

17. The method of claim 16, wherein said Vitamin A metabolite is an all-trans-retinoic acid.

18. The method of claim 16, wherein said Vitamin A agonist is a retinoic acid receptor agonist.

19. A method of treating a benign or malignant hyperproliferative epidermal pathology in a subject in need thereof, the method comprising:
administering to said subject a therapeutically effective amount of a first agent selected from the group consisting of nicotinamide, a nicotinamide agonist, a nicotinamide derivative, a nicotinamide metabolite and prodrugs thereof, in combination with a second agent selected from the group consisting of cyclic adenosine diphosphate-ribose (cADPR), a cADPR derivative, a cADPR metabolite, a cADPR agonist and prodrugs thereof.

20. The method of claim 19, wherein said hyperproliferative benign epidermal pathology is selected from the group consisting of psoriasis, ichythyiosis, common warts, keratoacanthoma, seborrhoic keratosis and seborrhea.

21. The method of claim 19, wherein said hyperproliferative malignant epidermal pathology is selected from the group consisting of squamous-cell carcinoma (SCC), basal cell carcinoma (BCC) and a non-melanoma skin cancer (NMSC).

22. The method of claim 19, further comprising:
administering to said subject, in combination with said first and second agents, a therapeutically effective amount of an agent selected from the group consisting of Vitamin D3, a Vitamin D3 metabolite, a Vitamin D3 agonist, a Vitamin D3 derivative and prodrugs thereof.

23. The method of claim 22, wherein said Vitamin D3 metabolite is 1α,25-dihydroxy-vitamin D3.

24. The method of claim 19, further comprising:
administering to said subject, in combination with said first and second agents, a therapeutically effective amount of an agent selected from the group consisting of Vitamin A, a Vitamin A metabolite, a Vitamin A agonist, a Vitamin A derivative and prodrugs thereof.

25. The method of claim 24, wherein said Vitamin A metabolite is an all-trans-retinoic acid.

26. The method of claim 24, wherein said Vitamin A agonist is a retinoic acid receptor agonist.

27. The method of claim 19, wherein said first agent is nicotinamide and said second agent is cADPR.

28. A method of treating a benign or malignant hyperproliferative epidermal pathology in a subject in need thereof, the method comprising:
administering to said subject a therapeutically effective amount of a first agent selected from the group consisting of nicotinamide, a nicotinamide agonist, a nicotinamide derivative, a nicotinamide metabolite and prodrugs thereof, in combination with a therapeutically effective amount of a second agent selected from the group consisting of Vitamin D3, a Vitamin D3 metabolite, a Vitamin D3 agonist, a Vitamin D3 derivative and prodrugs thereof.

29. The method of claim 28, wherein said hyperproliferative benign epidermal pathology is selected from the group consisting of psoriasis, ichythyiosis, common warts, keratoacanthoma, seborrhoic keratosis and seborrhea.

30. The method of claim 28, wherein said hyperproliferative malignant epidermal pathology is selected from the group consisting of squamous-cell carcinoma (SCC), basal cell carcinoma (BCC) and a non-melanoma skin cancer (NMSC).

31. The method of claim 28, wherein said first agent is nicotinamide and said second agent is a Vitamin D3 metabolite.

32. The method of claim 31, wherein said Vitamin D3 metabolite is 1α,25-dihydroxy-vitamin D3.

33. The method of claim 28, further comprising:
administering to said subject, in combination with said first and second agents, a therapeutically effective amount of an agent selected from the group consisting of an agent selected from the group consisting of cyclic adenosine diphosphate-ribose (cADPR), a cADPR derivative, a cADPR metabolite, a cADPR agonist and prodrugs thereof.

34. The method of claim 28, further comprising:
administering to said subject, in combination with said first and second agents, a therapeutically effective amount of an agent selected from the group consisting of Vitamin A, a Vitamin A metabolite, a Vitamin A agonist, a Vitamin A derivative and prodrugs thereof.

35. The method of claim 34, wherein said Vitamin A metabolite is an all-trans-retinoic acid.

36. The method of claim 34, wherein said Vitamin A agonist is a retinoic acid receptor agonist.

37. A method of treating a benign or malignant hyperproliferative epidermal pathology in a subject in need thereof, the method comprising:
administering to said subject a therapeutically effective amount of a first agent selected from the group consisting of nicotinamide, a nicotinamide agonist, a nicotinamide derivative, a nicotinamide metabolite and prodrugs thereof, in combination with a second agent selected from the group consisting of Vitamin A, a Vitamin A metabolite, a Vitamin A agonist, a Vitamin A derivative and prodrugs thereof.

38. The method of claim 37, wherein said hyperproliferative benign epidermal pathology is selected from the group consisting of psoriasis, ichythyiosis, common warts, keratoacanthoma, seborrhoic keratosis and seborrhea.

39. The method of claim 37, wherein said hyperproliferative malignant epidermal pathology is selected from the group consisting of squamous-cell carcinoma (SCC), basal cell carcinoma (BCC) and a non-melanoma skin cancer (NMSC).

40. The method of claim 37, wherein said first agent is nicotinamide and said second agent is a Vitamin A metabolite.

41. The method of claim 40, wherein said Vitamin A metabolite is an all-trans-retinoic acid.

42. The method of claim 37, further comprising:
administering to said subject, in combination with said first and second agents, a therapeutically effective amount of an agent selected from the group consisting of an agent selected from the group consisting cyclic adenosine diphosphate-ribose (cADPR), a cADPR derivative, a cADPR metabolite, a cADPR agonist and prodrugs thereof.

43. The method of claim 37, further comprising:
administering to said subject, in combination with said first and second agents, a therapeutically effective amount of an agent selected from the group consisting of Vitamin D3, a Vitamin D3 metabolite, a Vitamin D3 agonist, a Vitamin D3 derivative and prodrugs thereof.

44. The method of claim 43, wherein said Vitamin D3 metabolite is 1α,25 dihydroxy-vitamin D3.

45. A method of treating a benign or malignant hyperproliferative epidermal pathology in a subject in need thereof, the method comprising:
administering to said subject a therapeutically effective amount of a first agent selected from the group consisting of cyclic adenosine diphosphate-ribose (cADPR), a cADPR derivative, a cADPR metabolite, a cADPR agonist and prodrugs thereof, in combination with a second agent selected from the group consisting of Vitamin D3, a Vitamin D3 metabolite, a Vitamin D3 agonist, a Vitamin D3 derivative and prodrugs thereof.

46. The method of claim 45, wherein said hyperproliferative benign epidermal pathology is selected from the group consisting of psoriasis, ichythyiosis, common warts, keratoacanthoma, seborrhoic keratosis and seborrhea.

47. The method of claim 45, wherein said hyperproliferative malignant epidermal pathology is selected from the group consisting of squamous-cell carcinoma (SCC), basal cell carcinoma (BCC) and a non-melanoma skin cancer (NMSC).

48. The method of claim 45, wherein said first agent is cyclic adenosine diphosphate-ribose (cADPR) and said second agent is a Vitamin D3 metabolite.

49. The method of claim 48, wherein said Vitamin D3 metabolite is 1α25 dihydroxy-vitamin D3.

50. The method of claim 45, further comprising:
administering to said subject, in combination with said first and second agents, a therapeutically effective amount of an agent selected from the group consisting of nicotinamide, a nicotinamide derivative, a nicotinamide metabolite, a nicotinamide agonist and prodrugs thereof.

51. The method of claim 45, further comprising:
administering to said subject in combination with said first and second agents, a therapeutically effective amount of an agent selected from the group consisting of Vitamin A, a Vitamin A metabolite, a Vitamin A agonist, a Vitamin A derivative and prodrugs thereof.

52. The method of claim 51, wherein said Vitamin A metabolite is an all-trans-retinoic acid.

53. A method of treating a benign or malignant hyperproliferative epidermal pathology in a subject in need thereof, the method comprising:
administering to said subject a therapeutically effective amount of a first agent selected from the group consisting of cyclic adenosine diphosphate-ribose (cADPR), a cADPR derivative, a cADPR metabolite, a cADPR agonist and prodrugs thereof, in combination with a second agent selected from the group consisting of Vitamin A, a Vitamin A metabolite, a Vitamin A agonist, a Vitamin A derivative and prodrugs thereof.

54. The method of claim 53, wherein said hyperproliferative benign epidermal pathology is selected from the group consisting of psoriasis, ichythyiosis, common warts, keratoacanthoma, seborrhoic keratosis and seborrhea.

55. The method of claim 53, wherein said hyperproliferative malignant epidermal pathology is selected from the group consisting of squamous-cell carcinoma (SCC), basal cell carcinoma (BCC) and a non-melanoma skin cancer (NMSC).

56. The method of claim 53, wherein said first agent of cyclic adenosine diphosphate-ribose (cADPR) and said second agent is a Vitamin A metabolite.

57. The method of claim 56, wherein said Vitamin A metabolite is an all-trans-retinoic acid.

58. The method of claim 53, further comprising:
administering to said subject, in combination with said first and second agents, a therapeutically effective amount of an agent selected from the group consisting of nicotinamide, a nicotinamide agonist, a nicotinamide derivative, a nicotinamide metabolite and prodrugs thereof

59. The method of claim 53, further comprising:
administering to said subject, in combination with said first and second agents, a therapeutically effective amount of an agent selected from the group consisting of Vitamin D3, a Vitamin D3 metabolite, a Vitamin D3 agonist, a Vitamin D3 derivative and prodrugs thereof.

60. The method of claim 59, wherein said Vitamin D3 metabolite is 1α,25 dihydroxy-vitamin D3.

61. A method of increasing anti-oxidative properties of epidermal cells, the method comprising:
contacting said cells with an effective amount of an agent selected from the group consisting of nicotinamide, a nicotinamide agonist, a nicotinamide derivative, a nicotinamnide metabolite and prodrugs thereof.

62. The method of claim 61, wherein said agent is nicotinamide and said effective amount ranges between 1 mM and 50 mM.

63. A method of increasing anti-oxidative properties of epidermal cells, the method comprising:
contacting said cells with an effective amount of an agent selected from the group consisting of cyclic adenosine diphosphate-ribose (cADPR), a cADPR derivative, a cADPR metabolite, a cADPR agonist and prodrugs thereof.

64. A method of increasing anti-oxidative properties of epidermal cells, the method comprising:
contacting said cells with an effective amount of a first agent selected from the group consisting of nicotinamnide, a nicotinamide agonist, a nicotinamide derivative, a nicotinamide metabolite and prodrugs thereof, in combination with am effective amount of a second agent selected from the group consisting of cyclic adenosine diphosphate-ribose (cADPR), a cADPR derivative, a cADPR metabolite, a cADPR agonist and prodrugs thereof.

65. A method of increasing anti-oxidative properties of epidermal cells, the method comprising:
contacting said cells with an effective amount of a first agent selected from the group consisting of nicotinamide, a nicotinamide agonist, a nicotinamide derivative, a nicotinamide metabolite and prodrugs thereof, in combination with an effective amount of a second agent selected from the group consisting of Vitamin D3, a Vitamin D3 metabolite, a Vitamin D3 agonist, a Vitamin D3 derivative and prodrugs thereof.

66. A method of increasing anti-oxidative properties of epidermal cells, the method comprising:
contacting said cells with an effective amount of an agent selected from the group consisting of nicotinamide, a nicotinamide agonist, a nicotinamide derivative, a nicotinamide metabolite and prodrugs thereof, in combination with an effective amount of an agent selected from the group consisting of Vitamin A, a Vitamin A metabolite, a Vitamin A agonist, a Vitamin A derivative and prodrugs thereof.

67. A method of increasing anti-oxidative properties of epidermal cells, the method comprising:
contacting said cells with an effective amount of a first agent selected from the group consisting of cyclic adenosine diphosphate-ribose (cADPR), a cADPR derivative, a cADPR metabolite, a cADPR agonist and prodrugs thereof, in combination with an effective amount of a second agent selected from the group consisting of Vitamin D3, a Vitamin D3 metabolite, a Vitamin D3 agonist, a Vitamin D3 derivative and prodrugs thereof.

68. A method of increasing anti-oxidative properties of epidermal cells, the method comprising:
contacting said cells with an effective amount of a first agent selected from the group consisting of cyclic adenosine diphosphate-ribose (cADPR), a cADPR derivative, a cADPR metabolite, a cADPR agonist and prodrugs thereof in combination with an effective amount of a second agent selected from the group consisting of Vitamin A, a Vitamin A metabolite, a Vitamin A agonist, a Vitamin A derivative and prodrugs thereof.

69. A pharmaceutical, cosmetic or cosmeceutical composition, identified for use in the treatment of benign or malignant hyperproliferative epidermal pathology, and/or for use in the treatment of a condition whereby increasing anti-oxidative properties of epidermal cells is advantageous, comprising, as an active ingredient, a therapeutically effective amount of an agent selected from the group consisting of nicotinamide, a nicotinamide agonist, a nicotinamide derivative, a nicotinamide metabolite and prodrugs thereof, and a pharmaceutically, cosmetically or cosmeceutically acceptable carrier.

70. The pharmaceutical, cosmetic or cosmeceutical composition of claim 69, wherein said hyperproliferative benign epidermal pathology is selected from the group consisting of psoriasis, ichythyiosis, common warts, keratoacanthoma, seborrhoic keratosis and seborrhea.

71. The pharmaceutical, cosmetic or cosmeceutical composition of claim 69, wherein said hyperproliferative malignant epidermal pathology is selected from the group consisting of squamous-cell carcinoma (SCC), basal cell carcinoma (BCC) and a non-melanoma skin cancer (NMSC).

72. The pharmaceutical, cosmetic or cosmeceutical composition of claim 69, wherein said condition is aging.

73. The pharmaceutical, cosmetic or cosmeceutical composition of claim 69, wherein said condition is cancer.

74. The pharmaceutical, cosmetic or cosmeceutical composition of claim 69, wherein said agent is nicotinamide and said therapeutically effective amount ranges between 0.5 mM and 20 mM.

75. The pharmaceutical, cosmetic or cosmeceutical composition of claim 74, wherein said therapeutically effective amount ranges between 1 mM and 10 mM.

76. The pharmaceutical, cosmetic or cosmeceutical composition of claim 69, further comprising, in combination with said agent, a therapeutically effective amount of a second agent selected from the group consisting of cyclic adenosine diphosphate-ribose (cADPR), a cADPR derivative, a cADPR metabolite, a cADPR agonist and prodrugs thereof.

77. The pharmaceutical, cosmetic or cosmeceutical composition of claim 76, wherein said agent is nicotinamide and said second agent is cADPR.

78. The pharmaceutical, cosmetic or cosmeceutical composition of claim 77, wherein said therapeutically effective amount of said nicotinamide ranges between 1 mM and 10 mM and said therapeutically effective amount of said cADPR ranges between 10 μM and 100 μM.

79. The pharmaceutical, cosmetic or cosmeceutical composition of claim 69, further comprising, in combination with said agent, a therapeutically effective amount of a second agent selected from the group consisting of Vitamin D3, a Vitamin D3 metabolite, a Vitamin D3 agonist, a Vitamin D3 derivative and prodrugs thereof.

80. The pharmaceutical, cosmetic or cosmeceutical composition of claim 79, wherein said agent is nicotinamide and said second agent is a Vitamin D3 metabolite.

81. The pharmaceutical, cosmetic or cosmeceutical composition of claim 80, wherein said Vitamin D3 metabolite is 1α,25-dihydroxy-vitamin D3.

82. The pharmaceutical, cosmetic or cosmeceutical composition of claim 81, wherein said therapeutically effective amount of said nicotinamide ranges between 1 mM and 10 mM and said therapeutically effective amount of said 1α,25-dihydroxy-vitamin D3 ranges between 1 nM and 200 nM.

83. The pharmaceutical, cosmetic or cosmeceutical composition of claim 69, further comprising, in combination with said agent, a therapeutically effective amount of a second agent selected from tie group consisting of Vitamin A, a Vitamin A metabolite, a Vitamin A agonist, a Vitamin A derivative and prodrugs thereof.

84. The pharmaceutical, cosmetic or cosmeceutical composition of claim 83, wherein said agent is nicotinamide and said second agent is a Vitamin A metabolite.

85. The pharmaceutical, cosmetic or cosmeceutical composition of claim 84, wherein said Vitamin A metabolite is an all-trans-retinoic acid.

86. The pharmaceutical, cosmetic or cosmeceutical composition of claim 85, wherein said therapeutically effective amount of said nicotinamide ranges between 1 mM and 10 mM and said effective amount of said all-trans-retinoic acid ranges between 0.1 nM and 10 nM.

87. The pharmaceutical, cosmetic or cosmeceutical composition of claim 69, packaged in a container and identified in print on or in said container for use in treatment of a benign or a malignant hyperproliferative epidermal pathology.

88. The pharmaceutical, cosmetic or cosmeceutical composition of claim 69, packaged in a container and identified in print on or in said container for use in the treatment of a condition whereby increasing anti-oxidative properties of epidermal cells is advantageous.

89. A pharmaceutical, cosmetic or cosmeceutical composition, identified for use in the treatment of a benign or malignant hyperproliferative epidermal pathology, and/or for use in the treatment of a condition whereby increasing anti-oxidative properties of epidermal cells is advantageous, comprising, as an active ingredient, a therapeutically effective amount of an agent selected from the group consisting of cyclic adenosine diphosphate-ribose (cADPR), a cADPR derivative, a cADPR metabolite, a cADPR agonist and prodrugs thereof, and a pharmaceutically, cosmetically or cosmeceutically acceptable carrier.

90. The pharmaceutical, cosmetic or cosmeceutical composition of claim 89, wherein said hyperproliferative benign epidermal pathology is selected from the group consisting of psoriasis, ichythyiosis, common warts, keratoacanthoma, seborrhoic keratosis and seborrhea.

91. The pharmaceutical, cosmetic or cosmeceutical composition of claim 89, wherein said hyperproliferative malignant epidermal pathology is selected from the group consisting of squamous-cell carcinoma (SCC), basal cell carcinoma (BCC) and a non-melanoma skin cancer (NMSC).

92. The pharmaceutical, cosmetic or cosmeceutical composition of claim 89, wherein said condition is aging.

93. The pharmaceutical, cosmetic or cosmeceutical composition of claim 89, wherein said condition is cancer.

94. The pharmaceutical, cosmetic or cosmeceutical composition of claim 89, wherein said agent is cADPR and said therapeutically effective amount ranges between 10 μM and 100 μM.

95. The pharmaceutical, cosmetic or cosmeceutical composition of claim 89, further comprising, in combination with said agent, a therapeutically effective amount of a second agent selected from the group consisting of nicotinamide, a nicotinamide agonist, a nicotinamide derivative, a nicotinamide metabolite and prodrugs thereof.

96. The pharmaceutical, cosmetic or cosmeceutical composition of claim 95, wherein said agent is cADPR and said second agent is nicotinamide.

97. The pharmaceutical, cosmetic or cosmeceutical composition of claim 96, wherein said therapeutically effective amount of said nicotinamide ranges between 1 mM and to 10 mM and said therapeutically effective amount of said cADPR ranges between 10 μM and 100 μM.

98. The pharmaceutical, cosmetic or cosmeceutical composition of claim 89, further comprising, in combination with said agent, a therapeutically effective amount of a second agent selected from the group consisting of Vitamin D3, a Vitamin D3 metabolite, a Vitamin D3 agonist, a Vitamin D3 derivative and prodrugs thereof.

99. The pharmaceutical, cosmetic or cosmeceutical composition of claim 98, wherein said agent is cADPR and said second agent is a Vitamin D3 metabolite.

100. The pharmaceutical, cosmetic or cosmeceutical composition of claim 99, wherein said second Vitamin D3 metabolite is 1α,25-dihydroxy-vitamin D3.

101. The pharmaceutical, cosmetic or cosmeceutical composition of claim 89, further comprising, in combination with said agent, a therapeutically effective amount of a second agent selected from the group consisting of Vitamin A, a Vitamin A metabolite, a Vitamin A agonist, a Vitamin A derivative and prodrugs thereof.

102. The pharmaceutical, cosmetic or cosmeceutical composition of claim 101, wherein said agent is cADPR and said second agent is a Vitamin A metabolite.

103. The pharmaceutical, cosmetic or cosmeceutical composition of claim 102, wherein said Vitamin A metabolite is an all-trans-retinoic acid.

104. The pharmaceutical, cosmetic or cosmeceutical composition of claim 89, packaged in a container and identified in print on or in said container for use in treatment of a benign or a malignant hyperproliferative epidermal pathology.

105. The pharmaceutical, cosmetic or cosmeceutical composition of claim 89, packaged in a container and identified in print on or in said container for use in the treatment of a condition whereby increasing anti-oxidative properties of epidermal cells is advantageous.

106. A pharmaceutical, cosmetic or cosmeceutical composition, identified for use in the treatment of a benign or malignant hyperproliferative epidermal pathology, and/or for use in the treatment of a condition whereby increasing anti-oxidative properties of epidermal cells is advantageous, comprising, as a combination of active ingredients, a therapeutically effective amount of a first agent selected from the group consisting of nicotinamide, a nicotinamide agonist, a nicotinamide derivative, a nicotinamide metabolite and prodrugs thereof, a therapeutically effective amount of a second agent selected from the group consisting of cyclic adenosine diphosphate-ribose (cADPR), a cADPR derivative, a cADPR metabolite, a cADPR agonist and prodrugs thereof, and a pharmaceutically, cosmetically or cosmeceutically acceptable carrier.

107. The pharmaceutical, cosmetic or cosmeceutical composition of claim 106, wherein said hyperproliferative benign epidermal pathology is selected from the group consisting of psoriasis, ichythyiosis, common warts, keratoacanthoma, seborrhoic keratosis and seborrhea.

108. The pharmaceutical, cosmetic or cosmeceutical composition of claim 106, wherein said hyperproliferative malignant epidermal pathology is selected from the group consisting of squamous-cell carcinoma (SCC), basal cell carcinoma (BCC) and a non-melanoma skin cancer (NMSC).

109. The pharmaceutical, cosmetic or cosmeceutical composition of claim 106, wherein said condition is aging.

110. The pharmaceutical, cosmetic or cosmeceutical composition of claim 106, wherein said condition is cancer.

111. The pharmaceutical, cosmetic or cosmeceutical composition of claim 106, wherein said first agent is nicotinamide and said second agent is cADPR.

112. The pharmaceutical, cosmetic or cosmeceutical composition of claim 111, wherein said therapeutically effective amount of said nicotinamide ranges between 0.5 mM and 20 mM and said therapeutically effective amount of said cADPR ranges between 10 μM and 100 μM.

113. The pharmaceutical, cosmetic or cosmeceutical composition of claim 106, further comprising, in combination with said first and second agents, a therapeutically effective amount of an agent selected from the group consisting of Vitamin D3, a Vitamin D3 metabolite, a Vitamin D3 agonist, a Vitamin D3 derivative and prodrugs thereof.

114. The pharmaceutical, cosmetic or cosmeceutical composition of claim 106, further comprising, in combination with said first and second agents, a therapeutically effective amount of an agent selected from the group consisting of Vitamin A, a Vitamin A metabolite, a Vitamin A agonist, a Vitamin A derivative and prodrugs thereof.

115. The pharmaceutical, cosmetic or cosmeceutical composition of claim 106, packaged in a container and identified in print on or in said container for use in treatment of a benign or a malignant hyperproliferative epidermal pathology.

116. The pharmaceutical, cosmetic or cosmeceutical composition of claim 106, packaged in a container and identified in print on or in said container for use in the treatment of a condition whereby increasing anti-oxidative properties of epidermal cells is advantageous.

117. A pharmaceutical, cosmetic or cosmeceutical composition, identified for use in the treatment of a benign or malignant hyperproliferative epidermal pathology, and/or for use in the treatment of a condition whereby increasing anti-oxidative properties of epidermal cells is advantageous, comprising, as a combination of active ingredients, a therapeutically effective amount of a first agent selected from the group consisting of nicotinamide, a nicotinamide agonist, a nicotinamide derivative, a nicotinamide metabolite and prodrugs thereof, a therapeutically effective amount of a second agent selected from the group consisting of Vitamin D3, a Vitamin D3 metabolite, a Vitamin D3 agonist, a Vitamin D3 derivative and prodrugs thereof, and a pharmaceutically, cosmetically or cosmeceutically acceptable carrier.

118. The pharmaceutical, cosmetic or cosmeceutical composition of claim 117, wherein said hyperproliferative benign epidermal pathology is selected from the group consisting of psoriasis, ichythyiosis, common warts, keratoacanthoma, seborrhoic keratosis and seborrhea.

119. The pharmaceutical, cosmetic or cosmeceutical composition of claim 117, wherein said hyperproliferative malignant epidermal pathology is selected from the group consisting of squamous-cell carcinoma (SCC), basal cell carcinoma (BCC) and a non-melanoma skin cancer (NMSC).

120. The pharmaceutical, cosmetic or cosmeceutical composition of claim 117, wherein said condition is aging.

121. The pharmaceutical, cosmetic or cosmeceutical composition of claim 117, wherein said condition is cancer.

122. The pharmaceutical, cosmetic or cosmeceutical composition of claim 117, wherein said first agent is nicotinamide and said second agent is a Vitamin D3 metabolite.

123. The pharmaceutical, cosmetic or cosmeceutical composition of claim 122, wherein said Vitamin D3 metabolite is 1α,25-dihydroxy-vitamin D3.

124. The pharmaceutical, cosmetic or cosmeceutical composition of claim 123, wherein said therapeutically effective amount of said nicotinamide ranges between 1 mM and 10 mM and said therapeutically effective amount of said 1α,25-dihydroxy-vitamin D3 ranges between 1 nM and 200 nM.

125. The pharmaceutical, cosmetic or cosmeceutical composition of claim 117, further comprising, in combination with said first and second agents, a therapeutically effective amount of an agent selected from the group consisting of cyclic adenosine diphosphate-ribose (cADPR), a cADPR derivative, a cADPR metabolite, a cADPR agonist and prodrugs thereof.

126. The pharmaceutical, cosmetic or cosmeceutical composition of claim 117, further comprising, in combination with said first and second agents, a therapeutically effective amount of a third agent selected from the group consisting of Vitamin A, a Vitamin A metabolite, a Vitamin A agonist, a Vitamin A derivative and prodrugs thereof.

127. The pharmaceutical, cosmetic or cosmeceutical composition of claim 117, packaged in a container and identified in print on or in said container for use in treatment of a benign or a malignant hyperproliferative epidermal pathology.

128. The pharmaceutical, cosmetic or cosmeceutical composition of claim 117, packaged in a container and identified in print on or in said container for use in the treatment of a condition whereby increasing anti-oxidative properties of epidermal cells is advantageous.

129. A pharmaceutical, cosmetic or cosmeceutical composition, identified for use in the treatment of a benign or malignant hyperproliferative epidermal pathology, and/or for use in the treatment of a condition whereby increasing anti-oxidative properties of epidermal cells is advantageous, comprising, as a combination of active ingredients, a therapeutically effective amount of a first agent selected from the group consisting of nicotinamide, a nicotinamide agonist, a nicotinamide derivative, a nicotinamide metabolite and prodrugs thereof, a therapeutically effective amount of a second agent selected from the group consisting of Vitamin A, a Vitamin A metabolite, a Vitamin A agonists a Vitamin A derivative and prodrugs thereof, and a pharmaceutically, cosmetically or cosmeceutically acceptable carrier.

130. The pharmaceutical, cosmetic or cosmeceutical composition of claim 129, wherein said hyperproliferative benign epidermal pathology is selected from the group consisting of psoriasis, ichythyiosis, common warts, keratoacanthoma, seborrhoic keratosis and seborrhea.

131. The pharmaceutical, cosmetic or cosmeceutical composition of claim 129, wherein said hyperproliferative malignant epidermal pathology is selected from the group consisting of squamous-cell carcinoma (SCC), basal cell carcinoma (BCC) and a non-melanoma skin cancer (NMSC).

132. The pharmaceutical, cosmetic or cosmeceutical composition of claim 129, wherein said condition is aging.

133. The pharmaceutical, cosmetic or cosmeceutical composition of claim 129, wherein said condition is cancer.

134. The pharmaceutical, cosmetic or cosmeceutical composition of claim 129, wherein said first agent is nicotinamide and said second agent is a Vitamin A metabolite.

135. The pharmaceutical, cosmetic or cosmeceutical composition of claim 134, wherein said Vitamin A metabolite is an all-trans-retinoic acid.

136. The pharmaceutical, cosmetic or cosmeceutical composition of claim 135, wherein said therapeutically effective amount of said nicotinamide ranges between 1 mM and 10 mM and said therapeutically effective amount of said all-trans-retinoic acid ranges between 0.1 nM and 10 nM.

137. The pharmaceutical, cosmetic or cosmeceutical composition of claim 129, father comprising, in combination with said first and second agents, a therapeutically effective amount of an agent selected from the group consisting of cyclic adenosine diphosphate-ribose (cADPR), a cADPR derivative, a cADPR metabolite, a cADPR agonist and prodrugs thereof.

138. The pharmaceutical, cosmetic or cosmeceutical composition of claim 129, further comprising, in combination with said first and second agents, a therapeutically effective amount of a third agent selected from the group consisting of Vitamin D3, a Vitamin D3 metabolite, a Vitamin D3 agonist, a Vitamin D3 derivative and prodrugs thereof.

139. The pharmaceutical, cosmetic or cosmeceutical composition of claim 129, packaged in a container and identified in print on or in said container for use in treatment of a benign or a malignant hyperproliferative epidermal pathology.

140. The pharmaceutical, cosmetic or cosmeceutical composition of claim 129, packaged in a container and identified in print on or in said container for use in the treatment of a condition whereby increasing anti-oxidative properties of epidermal cells is advantageous.

141. A pharmaceutical, cosmetic or cosmeceutical composition, identified for use in the treatment of a benign or malignant hyperproliferative epidermal pathology, and/or for use in the treatment of a condition whereby increasing anti-oxidative properties of epidermal cells is advantageous, comprising, as a combination of active ingredients, a therapeutically effective amount of a first agent selected from the group consisting of cyclic adenosine diphosphate-ribose (cADPR), a cADPR derivative, a cADPR metabolite, a cADPR agonist and prodrugs thereof, a therapeutically effective amount of a second agent selected from the group consisting of Vitamin D3, a Vitamin D3 metabolite, a Vitamin D3 agonist, a Vitamin D3 derivative and prodrugs thereof, and a pharmaceutically, cosmetically or cosmeceutically acceptable carrier.

142. The pharmaceutical, cosmetic or cosmeceutical composition of claim 141, wherein said hyperproliferative benign epidermal pathology is selected from the group consisting of psoriasis, ichythyiosis, common warts, keratoacanthoma, seborrhoic keratosis and seborrhea.

143. The pharmaceutical, cosmetic or cosmeceutical composition of claim 141, wherein said hyperproliferative malignant epidermal pathology is selected from the group consisting of squamous-cell carcinoma (SCC), basal cell carcinoma (BCC) and a non-melanoma skin cancer (NMSC).

144. The pharmaceutical, cosmetic or cosmeceutical composition of claim 141, wherein said condition is aging.

145. The pharmaceutical, cosmetic or cosmeceutical composition of claim 141, wherein said condition is cancer.

146. The pharmaceutical, cosmetic or cosmeceutical composition of claim 141, wherein said first agent is cADPR and said second agent is a Vitamin D3 metabolite.

147. The pharmaceutical, cosmetic or cosmeceutical composition of claim 146, wherein said Vitamin D3 metabolite is 1α,25-dihydroxy-vitamin D3.

148. The pharmaceutical, cosmetic or cosmeceutical composition of claim 141, further comprising, in combination with said fist and second agents, a therapeutically effective amount of a third agent selected from the group consisting of nicotinamide, a nicotinamide agonist, a nicotinamide derivative, a nicotinamide metabolite and prodrugs thereof.

149. The pharmaceutical, cosmetic or cosmeceutical composition of claim 141, further comprising, in combination with said first and second agents, a therapeutically effective amount of a third agent selected from the group consisting of Vitamin A, a Vitamin A metabolite, a Vitamin A agonist, a Vitamin A derivative and prodrugs thereof.

150. The pharmaceutical, cosmetic or cosmeceutical composition of claim 141, packaged in a container and identified in print on or in said container for use in treatment of a benign or a malignant hyperproliferative epidermal pathology.

151. The pharmaceutical, cosmetic or cosmeceutical composition of claim 141, packaged in a container and identified in print on or in said container for use in the treatment of a condition whereby increasing anti-oxidative properties of epidermal cells is advantageous.

152. A pharmaceutical, cosmetic or cosmeceutical composition, identified for use in the treatment of benign or malignant hyperproliferative epidermal pathology, comprising, as a combination of active ingredients, a therapeutically effective amount of a first agent selected from the group consisting of cyclic adenosine diphosphate-ribose (cADPR), a cADPR derivative, a cADPR metabolite, a cADPR agonist and prodrugs thereof, a therapeutically effective amount of a second agent selected from the group consisting of Vitamin A, a Vitamin A metabolite, a Vitamin A agonist, a Vitamin A derivative and prodrugs thereof, and a pharmaceutically, cosmetically or cosmeceutically acceptable carrier.

153. The pharmaceutical, cosmetic or cosmeceutical composition of claim 152, wherein said hyperproliferative benign epidermal pathology is selected from the group consisting of psoriasis, ichythyiosis, common warts, keratoacanthoma, seborrhoic keratosis and seborrhea.

154. The pharmaceutical, cosmetic or cosmeceutical composition of claim 152, wherein said hyperproliferative malignant epidermal pathology is selected from the group consisting of squamous-cell carcinoma (SCC), basal cell carcinoma (BCC) and a non-melanoma skin cancer (NMSC).

155. The pharmaceutical, cosmetic or cosmeceutical composition of claim 152, wherein said condition is aging.

156. The pharmaceutical, cosmetic or cosmeceutical composition of claim 152, wherein said condition is cancer.

157. The pharmaceutical, cosmetic or cosmeceutical composition of claim 152, wherein said first agent is cADPR and said second agent is a Vitamin A metabolite.

158. The pharmaceutical, cosmetic or cosmeceutical composition of claim 157, wherein said Vitamin A metabolite is an all-trans-retinoic acid.

159. The pharmaceutical, cosmetic or cosmeceutical composition of claim 152, further comprising, in combination with said first and second agents, a therapeutically effective amount of a third agent selected from the group consisting of nicotinamide, a nicotinamide derivative, a nicotinamide netabolite, a nicotinamide agonist and prodrugs thereof.

160. The pharmaceutical, cosmetic or cosmeceutical composition of claim 152, further comprising, in combination with said first and second agents, a therapeutically effective amount of a third agent selected from the group consisting of Vitamin D3, a Vitamin D3 metabolite, a Vitamin D3 agonist, a Vitamin D3 derivative and prodrugs thereof.

161. The pharmaceutical, cosmetic or cosmeceutical composition of claim 152, packaged in a container and identified in print on or in said container for use in treatment of a benign or a malignant hyperproliferative epidermal pathology.

162. The pharmaceutical, cosmetic or cosmeceutical composition of claim 152, packaged in a container and identified in print on or in said container for use in the treatment of a condition whereby increasing anti-oxidative properties of epidermal cells is advantageous.

163. A method of inhibiting proliferation of benign or malignant hyperproliferative epidermal cells, the method comprising:
contacting said cells with a therapeutically effective amount of all agent selected from the group consisting of nicotinamide, a nicotinamide agonist, a nicotinamide derivative, a nicotinamide metabolite and prodrugs thereof.

164. A method of inhibiting proliferation of benign or malignant hyperproliferative epidermal cells, the method comprising:
contacting said cells with a therapeutically effective amount of an agent selected from the group consisting of cyclic adenosine diphosphate-ribose (cADPR), a cADPR derivative, a cADPR metabolite, a cADPR agonist and prodrugs thereof.

165. A method of inhibiting proliferation of benign or malignant hyperproliferative epidermal cells, the method comprising:
contacting said cells with a therapeutically effective amount of a first agent selected from the group consisting of nicotinamide, a nicotinamide agonist, a nicotinamide derivative, a nicotinamide metabolite and prodrugs thereof, in combination with a therapeutically effective amount of a second agent selected from the group consisting of cyclic adenosine diphosphate-ribose (cADPR), a cADPR derivative, a cADPR metabolite, a cADPR agonist and prodrugs thereof.

166. A method of inhibiting proliferation of benign or malignant hyperproliferative epidermal cells, the method comprising:
contacting said cells with a therapeutically effective amount of a first agent selected from the group consisting of nicotinamide, a nicotinamide agonist, a nicotinamide derivative, a nicotinamide metabolite and prodrugs thereof, in combination with a therapeutically effective amount of a second agent selected from the group consisting of Vitamin D3, a Vitamin D3 metabolite, a Vitamin D3 agonist, a Vitamin D3 derivative and prodrugs thereof.

167. A method of inhibiting proliferation of benign or malignant hyperproliferative epidermal cells, the method comprising:
contacting said cells with a therapeutically effective amount of a first agent selected from the group consisting of nicotinamide, a nicotinamide agonist, a nicotinamide derivative, a nicotinamide metabolite and prodrugs thereof, in combination with a therapeutically effective amount of a second agent selected from the group consisting of Vitamin A, a Vitamin A metabolite, a Vitamin A agonist, a Vitamin A derivative and prodrugs thereof.

168. A method of inhibiting proliferation of benign or malignant hyperproliferative epidermal cells, the method comprising:
contacting said cells with a therapeutically effective amount of a first agent selected from the group consisting of cyclic adenosine diphosphate-ribose (cADPR), a cADPR derivative, a cADPR metabolite, a cADPR agonist and prodrugs thereof, in combination with a therapeutically effective amount of a second agent selected from the group consisting of Vitamin D3, a Vitamin D3 metabolite, a Vitamin D3 agonist, a Vitamin D3 derivative and prodrugs thereof.

169. A method of inhibiting proliferation of benign or malignant hyperproliferative epidermal cells, the method comprising;
contacting said cells with a therapeutically effective amount of a first agent selected from the group consisting of cyclic adenosine diphosphate-ribose (cADPR), a cADPR derivative, a cADPR metabolite, a cADPR agonist and prodrugs thereof, in combination with a therapeutically effective amount of a second agent selected from the group consisting of Vitamin A, a Vitamin A metabolite, a Vitamin A agonist, a Vitamin A derivative and prodrugs thereof.

170. A pharmaceutical, cosmetic or cosmeceutical kit, identified in print for use in the treatment of a benign or malignant hyperproliferative epidermal pathology, and/or for use in the treatment of a condition whereby increasing anti-oxidative properties of epidermal cells is advantageous, comprising, as an active ingredient an agent selected from the group consisting of nicotinamide, a nicotinamide agonist, a nicotinamide derivative, a nicotinamide metabolite and prodrugs thereof.

171. A pharmaceutical, cosmetic or cosmeceutical kit, identified in print for use in the treatment of a benign or malignant hyperproliferative epidermal pathology, and/or for use in the treatment of a condition whereby increasing anti-oxidative properties of epidermal cells is advantageous, comprising, as an active ingredient an agent selected from the group consisting of cyclic adenosine diphosphate-ribose (cADPR), a cADPR derivative, a cADPR metabolite, a cADPR agonist and prodrugs thereof.

172. A pharmaceutical, cosmetic or cosmeceutical kit, identified in print for use in the treatment of a benign or malignant hyperproliferative epidermal pathology, and/or for use in the treatment of a condition whereby increasing anti-oxidative properties of epidermal cells is advantageous, comprising, a first agent selected from the group consisting of nicotinamide, a nicotinamide agonist, a nicotinamide derivative, a nicotinamide metabolite and prodrugs thereof, and a second agent selected from the group consisting of cyclic adenosine diphosphate-ribose (cADPR), a cADPR derivative, a cADPR metabolite, a cADPR agonist and prodrugs thereof, said first agent and said second agent are individually packaged within the pharmaceutical, cosmetic or cosmeceutical kit.

173. A pharmaceutical, cosmetic or cosmeceutical kit, identified in print for use in the treatment of a benign or malignant hyperproliferative epidermal pathology, and/or for use in the treatment of a condition whereby increasing anti-oxidative properties of epidermal cells is advantageous, comprising, a first agent selected from the group consisting of nicotinamide, a nicotinamide agonist, a nicotinamide derivative, a nicotinamide metabolite and prodrugs thereof, and a second agent selected from the group consisting of Vitamin D3, a Vitamin D3 metabolite, a Vitamin D3 agonist, a Vitamin D3 derivative and prodrugs thereof, said first agent and said second agent are individually packaged within the pharmaceutical, cosmetic or cosmeceutical kit.

174. A pharmaceutical, cosmetic or cosmeceutical kit, identified in print for use in the treatment of a benign or malignant hyperproliferative epidermal pathology, and/or for use in the treatment of a condition whereby increasing anti-oxidative properties of epidermal cells is advantageous, comprising, a first agent selected from the group consisting of nicotinamide, a nicotinamide agonist, a nicotinamide derivative, a nicotinamide metabolite and prodrugs thereof, and a second agent selected from the group consisting of Vitamin A, a Vitamin A metabolite, a Vitamin A agonist, a Vitamin A derivative and prodrugs thereof, said first agent and said second agent are individually packaged within the pharmaceutical, cosmetic or cosmeceutical kit.

175. A pharmaceutical, cosmetic or cosmeceutical kit, identified in print for use in the treatment of a benign or malignant hyperproliferative epidermal pathology, and/or for use in the treatment of a condition whereby increasing anti-oxidative properties of epidermal cells is advantageous, comprising, a first agent selected from the group consisting of cyclic adenosine diphosphate-ribose (cADPR), a cADPR derivative, a cADPR metabolite, a cADPR agonist and prodrugs thereof, and a second agent selected from the group consisting of Vitamin D3, a Vitamin D3 metabolite, a Vitamin D3 agonist, a Vitamin D3 derivative and prodrugs thereof, said first agent and said second agent are individually packaged within the pharmaceutical, cosmetic or cosmeceutical kit.

176. A pharmaceutical, cosmetic or cosmeceutical kit, identified in print for use in the treatment of a benign or malignant hyperproliferative epidermal pathology, for use in the treatment of a condition whereby increasing anti-oxidative properties of epidermal cells is advantageous and/or for use in anti-cancer protection, comprising, a first agent selected from the group consisting of cyclic adenosine diphosphate-ribose (cADPR), a cADPR derivative, a cADPR metabolite, a cADPR agonist and prodrugs thereof, and a second agent selected from the group consisting of Vitamin A, a Vitamin A metabolite, a Vitamin A agonist, a Vitamin A derivative and prodrugs thereof, said first agent and said second agent are individually packaged within the pharmaceutical, cosmetic or cosmeceutical kit.
